(19) United States
(12) Patent Application Publication
(10) Pub. No.: US 2025/0018067 A1
(43) Pub. Date: Jan. 16, 2025

(54) HAFNIUM (IV) OXIDE NANOPARTICLES AND AQUEOUS COMPOSITIONS THEREOF

(71) Applicants: UNIVERSITÄT BASEL, Basel (CH); UNIVERSITEIT GENT, Gent (BE)

(72) Inventors: Loren DEBLOCK, Gent (BE); Jonathan DE ROO, Basel (CH)

(73) Assignees: UNIVERSITÄT BASEL, Basel (CH); UNIVERSITEIT GENT, Gent (BE)

(21) Appl. No.: 18/711,699

(22) PCT Filed: Nov. 23, 2022
(86) PCT No.: PCT/EP2022/083006

§ 371 Date: May 20, 2024

(30) Foreign Application Priority Data
Nov. 23, 2021 (EP) 21210057.2
May 24, 2022 (EP) 22175125.8

(51) Int. Cl. A61K 49/04; A61K 9/51; A61K 47/54

(52) U.S. Cl. CPC A61K 49/04; A61K 9/5123; A61K 47/54

(57) ABSTRACT

The invention relates to a hafnium (IV) oxide (HfO₂) nanoparticle nanocrystal comprising or having a diameter equal or less than (≤) 15 nm, stabilized by a plurality of dispersant molecules attached to its surface. The dispersant molecules comprise of a catechol or gallol surface adsorption moiety and an oligo (ethyleneglycol) moiety. The invention further relates to a composition of such nanoparticles in stable colloidal suspension at physiological pH, and the use of the composition in medical treatment and diagnostic applications, particularly to enhance radiotherapy and as an X ray contrast agent. In yet another aspect, the invention provides a method for manufacturing compositions and nanoparticles according to the invention.

This application claims the right of priority of European Patent Applications EP21210057.2 filed 23 Nov. 2021, and EP22175125.8 filed 24 May 2022, both of which are incorporated by reference herein.

### FIELD

The present invention relates to hafnium (IV) oxide nanoparticles that are associated with surface-adsorbed oligo (ethylenglycol) moieties. The invention further relates to compositions comprising the nanoparticles according to the invention for use as a pharmaceutical or a diagnostic.

### BACKGROUND OF THE INVENTION

Colloidal nanocrystals (NCs) have been considered for a multitude of biomedical applications, e.g., bio-imaging, drug delivery, photothermal therapy, and radiotherapy enhancement. These NCs are typically hybrid objects, consisting of an inorganic core capped with organic ligands. Ligands determine the interactions between NC and solvent and the stability of the nanocolloid. In the case of biomedical applications, controlling the NC surface chemistry is key since it will play a role in particle agglomeration, cellular uptake, protein repelling or adsorption, cytotoxicity, circulation time and targeted approaches. While surface chemistry is important for all types of NCs (chalcogenides, pnictides, halides, and metal NCs), here is not one solution that fits all. For example, thiolates and thiols have a strong binding affinity to Au and CdSe NCs but interact poorly with metal oxide NCs.

Metal oxide NCs have been particularly successful in nanomedicine. Three types of inorganic NCs have achieved clinical translation and two of them are oxides; iron oxide and hafnium oxide (Min, Y. Chemical Reviews 2015, 115 (19), 11147-11190). These particles are often first synthesized in nonpolar solvents, stabilized by surfactants (usually with a carboxylate or phosphonate head group and an aliphatic tail). Carboxylic acids (e.g., oleic acid) dissociate on the metal oxide surface, with carboxylates binding to surface metals sites and protons to surface oxygen atoms. This binding motif is written as NC(XX′) since both proton and carboxylate are X-type ligands. In nonpolar solvents, carboxylic acids are quantitatively exchanged by phosphonic acids in an X-for-X ligand exchange process. Indeed, phosphonic acids are very strong ligands for oxide surfaces. In contrast, catechol was found to be a rather weak ligand, only able to exchange a minor fraction of oleic acid. There is thus a clear order in binding strength in nonpolar solvents: catechol<carboxylic acid<phosphonic acid.

In aqueous (or other polar) environments, the order is less clear, and further complicated by variable factors such as pH and salt concentration. For example, carboxylic acids are frequently used to stabilize metal oxide NCs in water. They are able to provide colloidal stability in static systems with no competing ligands, but not in phosphate buffered saline (PBS) or cell culture media. The binding affinity is significantly increased for multidentate carboxylate ligands such as polymers. In general, literature reports agree that carboxylic acids are the weakest ligands in water, weaker than phosphonic acids or catechols. The literature is however inconclusive as to whether phosphonic acids or catechols are the best ligands. Despite the common usage of phosphonic acids and catechols in metal oxide NC functionalization, there is no clear consensus on the relative binding affinity. Furthermore, a direct link between ligand binding equilibria and the final colloidal stability of the NCs is usually not made.

Based on the above-mentioned state of the art, the objective of the present invention is to provide means and methods to provide better nanoparticle compositions for use in medical applications. This objective is attained by the subject-matter of the independent claims of the present specification, with further advantageous embodiments described in the dependent claims, examples, figures and general description of this specification.

### SUMMARY OF THE INVENTION

One aspect of the invention relates to a nanoparticle comprising a nanocrystal comprising or essentially consisting of hafnium (IV) oxide (HfO₂) having a diameter equal or less than(s) 15 nm, the nanocrystal being stabilized by a plurality of dispersant molecules attached to the surface of the nanocrystal.

The dispersant molecules comprise or essentially consist of a surface adsorption moiety selected from the group comprising a catechol or a gallol, and an oligo (ethyleneglycol) moiety.

The invention, in another aspect, relates to a composition of nanoparticles as described herein. The composition provides nanoparticles in stable colloidal suspension at physiological pH.

Another aspect of the invention relates to the use of the nanoparticles and compositions described herein in treatment and diagnostic applications, particularly to enhance radiotherapy and as an X ray contrast agent.

The present invention also relates a pharmaceutical composition comprising a nanoparticle or nanoparticle suspension according the present and at least one pharmaceutically acceptable carrier, diluent or excipient.

In yet another aspect, the invention provides a method for manufacturing compositions and nanoparticles according to the invention.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term nitrodopamine refers to a dopamine moiety bearing a nitro function on the dihydroxyphenyl ring of the dopamine moiety. One particular example for nitrodopamine is the following formula:

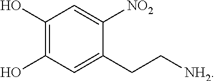 [SMILES: NCCc1cc(O)c(O)cc1[N+](=O)[O-]]

The term nitroDOPA refers to a DOPA moiety bearing a nitro function on the dihydroxyphenyl ring of the DOPA (dihydroxyphenylalanine moiety). One particular example for nitroDOPA is the following formula:

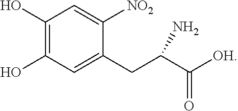 [SMILES: N[C@@H](Cc1cc(O)c(O)cc1[N+](=O)[O-])C(=O)O]

The term mimosine refers to (2S)-2-Amino-3-(3-hydroxy-4-oxopyridin-1-yl) propanoic acid of the following formula:

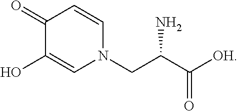 [SMILES: N[C@@H](Cn1ccc(=O)c(O)c1)C(=O)O]

An oligo-ethyleneglycol moiety as used herein relates to a chain described by the formula (CH₂—CH₂—O)ₙCH₃ wherein n is an integer ranging from 1 to 15, particularly with n ranging from 2 to 12, more particularly with n ranging from 2 to 5.

In the work on which the present specification is based, the inventors set out to unambiguously establish the binding affinity order and provide the correct surface chemistry for optimal application in nanomedicine. The inventors chose HfO₂ NCs as their model system for two reasons: (1) it is a relevant material in nanomedicine, and (2) it is compatible with solution Nuclear Magnetic Resonance (NMR) spectroscopy. The latter has proven to be a very powerful tool to study nanocrystal surface chemistry. Unfortunately, iron oxide NCs interfere with magnetic fields and cannot be studied in NMR. HfO₂ NCs are thus an ideal starting point, also because their surface chemistry has already been extensively studied in nonpolar solvents using NMR spectroscopy. First, the inventors evaluated the ligand exchange of the native carboxylic acid ligands for phosphonic acid and catechol ligands, using ¹H and ³¹P NMR spectroscopy. Importantly, they use the same poly(ethyleneglycol) ligand chain for all three binding group, thus ensuring that results can directly be compared. Next, they assess the influence of solvent (methanol vs water) and the pH on ligand binding. Furthermore, the inventors used NMR and Dynamic Light Scattering (DLS) to determine the colloidal stability provided by the different ligand types in aqueous and buffer environments, and directly correlate this to ligand binding dynamics. Finally, the inventors constructed a colloidal stability map, showing which binding group provides colloidal stability in function of pH. This practical guide will help researchers in designing future surface chemistries.

### DETAILED DESCRIPTION OF THE INVENTION

A first aspect of the invention relates to a nanoparticle comprising a nanocrystal core and an organic stabilizer adsorbed to the nanocrystal core, which facilitates the stability of the particle in aqueous solutions at physiological pH.

The nanoparticle is composed of a nanocrystal comprising or essentially consisting of hafnium (IV) oxide (HfO₂) having a diameter equal or less than ($) 15 nm, and a plurality of dispersant molecules attached to the surface of the nanocrystal. The dispersant molecules comprise or essentially consist of
i. a surface adsorption moiety selected from the group comprising a catechol or a gallol, particularly a 1,2-hydroxy-4-nitrophenyl moiety; and
ii. an oligo (ethyleneglycol) moiety.

One non-limiting example of such dispersant molecule is the dopamine-MEEAA adduct (MEEAA is 2-[2-(2-methoxyethoxy)ethoxy]acetic acid):

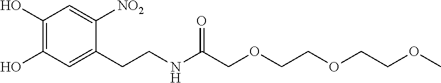 [SMILES: COCCOCCOCC(=O)NCCc1cc(O)c(O)cc1[N+](=O)[O-]]

In certain embodiments, the dopamine-oligo (ethylenglycol) dispersant molecules can be described more generally by the general formula

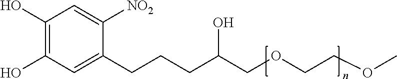

with n being selected from 1, 2, 3, 4, 5, 6.

In certain embodiments, the dopamine-oligo (ethylenglycol) dispersant molecule can be described more generally by the general formula

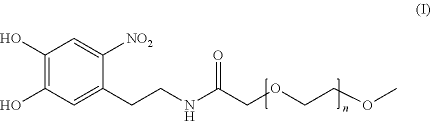

with n being selected from 1, 2, 3, 4, 5, 6.

In certain embodiments, the particle consists of pure hafnium (IV) oxide (HfO₂). This is chemically the most easily accessible nanocrystal. It is however conceivable that the HfO₂ is only part of the particle; for example, Hf metal might constitute the core and oxide the surface of the particle. A higher content of Hf metal is expected to provide even higher contrast.

In certain embodiments, the nanocrystal has a diameter of ≤6 nm. In particular embodiments, the diameter is ≤4 nm. In more particular embodiments, wherein the diameter is ≤3.5 nm. The smaller the diameter, the higher the likelihood that the particles will easily diffuse in the body, and will be subject to renal clearance.

In certain embodiments, the nanocrystal has a diameter of ≤15 nm. The inventors estimate that particles up to about 15 nm in diameter provide the advantages of the invention in use as a CT contrast agent; the stability of the material depends on the length of the dispersant (oligoethyleneglycol) chain. If the chain is longer, also larger particles can be stabilized.

In certain embodiments, the nanocrystal is characterized by an aspect ratio of 0.5 to 0.9. The particles obtained by the inventors had approximately a rice grain shape. Uniformly shaped particles are expected to be physiologically more acceptable.

In certain embodiments, the dispersant molecule has a molecular mass of ≤500 g/mol. In certain particular embodiments, the dispersant molecule has a molecular mass of ≤400 g/mol.

In certain particular embodiments, the dispersant molecule comprises, particularly consists of,
i. a surface adsorption moiety selected from nitrodopamine, nitroDOPA, DOPA, dopamine, mimosine and
ii. an (CH₂—CH₂—O)ₙCH₃ moiety, with n being an integer selected from 2, 3, 4 and 5.

In certain particular embodiments, the dispersant molecule is described by a general formula

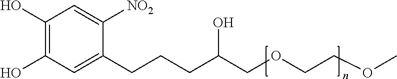

with n being selected from 1, 2, 3, 4, 5, 6.

In certain particular embodiments, the dispersant molecule is described by a general formula

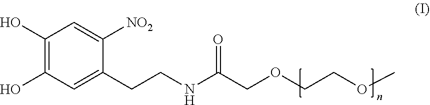

with n being selected from 1, 2, 3, 4, 5, 6.

In certain embodiments, the nanoparticle comprises an additional dispersant molecule, wherein the additional dispersant molecule is selected from the group consisting of

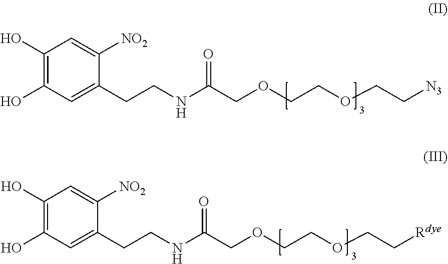

wherein R^{dye} is a fluorescent dye, optionally linked by covalent bond to the nitrodopamine moiety through a linker having 1 to 25 atoms of order number 12 or higher.

In certain particular embodiments, the additional dispersant molecule is described by a general formula

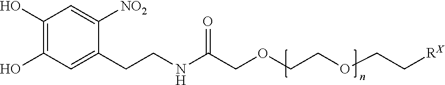

with n being selected from 1, 2, 3, 4, 5, 6, and wherein Rx is selected from the group consisting of a dye molecule (particularly a fluorescent dye molecule), and a functional chemical group facilitating a reaction with a dye molecule (particularly a functional chemical group selected from N₃, NH₂, OH, CCH (ethinyl)).

The term fluorescent dye in the context of the present specification relates to a small molecule capable of fluorescence in the visible or near infrared spectrum. Examples for fluorescent dye molecules or labels presenting a visible color include, without being restricted to, fluorescein isothiocyanate (FITC), rhodamine, allophycocyanine (APC), peridinin chlorophyll (PerCP), phycoerithrin (PE), alexa Fluors (Life Technologies, Carlsbad, CA, USA), dylight fluors (Thermo Fisher Scientific, Waltham, MA, USA) ATTO Dyes (ATTO-TEC GmbH, Siegen, Germany), BODIPY Dyes (4,4-difluoro-4-bora-3a,4a-diaza-s-indacene based dyes), 800CW dyes (indolium-based dyes) and the like.

In certain embodiments, the dispersant is

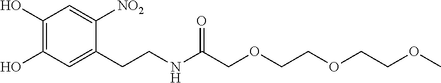 [SMILES: COCCOCCOCC(=O)NCCc1cc(O)c(O)cc1[N+](=O)[O-]]

Alternatives to the oligoethylene glycol moiety used herein include, but are not limited to, oligoglycerol and oligooxazoline chains.

In certain particular embodiments, the density of dispersant molecules on the nanocrystal is 0.5-5 per nm².

In certain particular embodiments, the nanoparticle comprises a dye molecule for optical localisation.

Another aspect of the invention relates to a composition comprising a plurality of nanoparticles according to the aspect and embodiments discussed above. In certain embodiments, the composition is a stable aqueous colloidal suspension.

In certain embodiments, the composition has a pH from pH 6 to pH 10. In certain particular embodiments, the composition has a pH ranging from pH 6.5 to pH 8.0. This is, to the knowledge of the inventors, the first time such ultrasmall HfO₂ particles have been provided in stable aqueous solution at physiological pH.

In certain embodiments, the composition is stable at neutral or basic pH values, particularly between pH 6 and pH 8.

In certain embodiments, 80% of the nanoparticles have a diameter between 2.0 nm and 5.0 nm. In certain particular embodiments, 85% of the nanoparticles have a diameter between 2.5 nm and 4.5 nm.

The compositions can be described, in respect of their size inhomogeneity, by size dispersion or the sigma used to fit the distribution to a Gaussian distribution. Exemplary parameters include, but are not limited to: Mean diameter=2.6 nm; Sigma=0.2 nm; size dispersion=7.7%.

The compositions can be described by their zeta potential. The zeta potential is a function of the pH; the inventors found that for the relevant pH range it is always negative.

Another aspect of the invention relates to the composition according to the invention, in any of its aspects or embodiments, for use in medicine. Several uses for HfO₂ particles have been described, prominently the use as a radiotherapy enhancing agent (radiosensitizer) (Maggiorella et al., Future Oncol. 2012 September; 8 (9): 1167-81) and the use as a computed tomography (CT) contrast agent (McGinnity, Nanoscale, 2016, 8, 13627-13637).

In certain embodiments, the concentration (v/v) of the nanoparticle inside the composition ranges from 15 μmol/L to 1000 μmol/L. In certain embodiments, the concentration (v/v) of the nanoparticle inside the composition ranges from 15 μmol/L to 500 μmol/L. In certain embodiments, the concentration (v/v) of the nanoparticle inside the composition ranges from 15 μmol/L to 250 μmol/L.

Yet another aspect of the invention relates to a method to manufacture a composition according to the invention. This method comprises the steps of:
a. providing a suspension of hafnium (IV) oxide (HfO₂) nanocrystals stabilized by a carboxylic acid ligand in aqueous medium;
b. adding to the suspension an alkaline solution of a dispersant molecule, wherein the dispersant molecule as described herein is composed of
   i. a surface adsorption moiety having two aromatic OH hydroxide functions at neutral pH, the surface adsorption moiety being selected from the group comprising a catechol or a gallol, particularly a 1,2-hydroxy-4-nitrophenyl moiety; and
   ii. an oligo (ethyleneglycol) moiety.

The pH of the alkaline solution of the dispersant molecule is chosen such that both aromatic hydroxide functions are deprotonated in the alkaline solution.
c. Subsequently, the pH of the suspension is adjusted to physiological pH and
d. optionally, the composition is separated or isolated. Particularly useful protocols for separation include size exclusion/spin filtration.
e. Another optional step includes sonication to resuspend agglomerates.

One carboxylic acid useful as a stabilizer in the first step of the method is MEEAA. This ligand binds poorly but good enough to disperse the particles in water initially.

In one example, the spin filtration step proceeds as follows: An NC suspension containing maximum 50 mg material dissolved in 2 ml solvent is transferred to a pre-rinsed Sartorius Vivaspin (30000 MWCO) spin-filtration tube via a 0.2 μm syringe filter. The suspension is diluted to a volume of 20 ml with Milli-Q water, the solution was then allowed to spin in a centrifuge for 30 mins at 2100 rcf. A minimum of 2 spin filtration cycles using Milli-Q water were performed until the filtrate was colorless. The concentrate was collected, evaporated and suspended in H2O, 30 minutes of sonication was performed to ensure that all agglomerates were resuspended and to minimize insoluble.

### Medical Treatment

Similarly, within the scope of the present invention is a method or treating a condition susceptible to radiotherapy, particularly cancer, in a patient in need thereof, comprising administering to the patient a composition according to the above description.

### Pharmaceutical Compositions, Administration/Dosage Forms and Salts

According to one aspect of the compound according to the invention, the nanoparticle or nanoparticle suspension according to the invention is provided as a pharmaceutical composition, pharmaceutical administration form, or pharmaceutical dosage form.

In certain embodiments of the invention, the nanoparticle or nanoparticle suspension of the present invention is typically formulated into pharmaceutical dosage forms to provide an easily controllable dosage of the drug and to give the patient an elegant and easily handleable product.

Similarly, a dosage form for the prevention or treatment of cancer is provided, comprising a nanoparticle or nanoparticle suspension according to any of the above aspects or embodiments of the invention.

The invention further encompasses a pharmaceutical composition comprising a nanoparticle or nanoparticle suspension of the present invention, and a pharmaceutically acceptable carrier. In further embodiments, the composition comprises at least two pharmaceutically acceptable carriers, such as those described herein.

Certain embodiments of the invention relate to a dosage form for parenteral administration, such as subcutaneous, intravenous, intrahepatic or intramuscular injection forms. Optionally, a pharmaceutically acceptable carrier and/or excipient may be present.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

### Method of Manufacture and Method of Treatment According to the Invention

The invention further encompasses, as an additional aspect, the use of a nanoparticle or nanoparticle suspension as identified herein, as specified in detail above, for use in a method of manufacture of a medicament. In particular embodiments, the medicament is provided for the radiotherapy, particularly in cancer, or as a contrast agent.

Wherever alternatives for single separable features are laid out herein as “embodiments”, it is to be understood that such alternatives may be combined freely to form discrete embodiments of the invention disclosed herein.

The invention further encompasses the following items.

### Items

1. A nanoparticle comprising
   a. a nanocrystal comprising or essentially consisting of hafnium (IV) oxide (HfO₂) having a diameter equal or less than ($) 15 nm, and
   b. a plurality of dispersant molecules attached to the surface of said nanocrystal, the dispersant molecules comprising or essentially consisting of
      i. a surface adsorption moiety selected from the group comprising a catechol or a gallol, particularly a 1,2-hydroxy-4-nitrophenyl moiety; and
      ii. an oligo (ethyleneglycol) moiety.
2. The nanoparticle according to item 1, wherein the nanocrystal has a diameter of ≤6 nm, particularly wherein the diameter is $4 nm, more particularly wherein the diameter is ≤3.5 nm.
3. The nanoparticle according to item 1 or 2, wherein the nanocrystal is characterized by an aspect ratio of 0.5 to 0.9.
4. The nanoparticle according to any one of items 1 to 3, wherein the dispersant molecule has a molecular mass of ≤500 g/mol, particularly of ≤400 g/mol.
5. The nanoparticle according to any one of items 1 to 4, wherein the dispersant molecule comprises, particularly consists of,
   i. a surface adsorption moiety selected from nitrodopamine, nitroDOPA, DOPA, dopamine, mimosine and
   ii. an (CH₂—CH₂—O)ₙCH₃ moiety, with n being an integer selected from 2, 3, 4 and 5; particularly wherein the dispersant molecule is

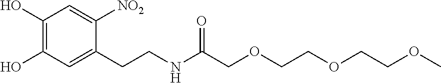 [SMILES: COCCOCCOCC(=O)NCCc1cc(O)c(O)cc1[N+](=O)[O-]]

6. The nanoparticle according to any one of items 1 to 5, wherein the density of dispersant molecules on the nanocrystal is 0.5-5 per nm².
7. The nanoparticle according to any one of items 1 to 6, wherein the nanoparticle comprises a dye molecule for optical localisation.
8. A composition comprising a plurality of nanoparticles according to any one of items 1 to 5.
9. The composition according to item 8, wherein the composition is a stable aqueous suspension.
10. The composition according to item 9, wherein the composition has a pH from pH 6 to pH 10, particularly a pH ranging from pH 6.5 to pH 8.0.
11. The composition according to item 7 or 8, wherein 80% of the nanoparticles have a diameter between 2.0 nm and 5.0 nm, particularly wherein 85% of the nanoparticles have a diameter between 2.5 nm and 4.5 nm.
12. A composition according to any one of items 8 to 11 for use in medicine.
13. A composition according to any one of items 8 to 11 for use as a radiotherapy enhancing agent (radiosensitizer).
14. A composition according to any one of items 8 to 11 for use as a computed tomography (CT) contrast agent.
15. A method to manufacture a composition according to any one of items 8 to 11, comprising
   a. providing a suspension of hafnium (IV) oxide (HfO₂) nanocrystals stabilized by a carboxylic acid ligand, particularly by MEEAA, in aqueous medium;
   b. adding to the suspension an alkaline solution of a dispersant molecule, wherein the dispersant molecule is composed of
      i. a surface adsorption moiety comprising two aromatic hydroxide functions, the surface adsorption moiety being selected from the group comprising a catechol or a gallol, particularly a 1,2-hydroxy-4-nitrophenyl moiety; and
      ii. an oligo (ethyleneglycol) moiety;
         wherein both aromatic hydroxide functions are deprotonated in the alkaline solution;
   c. adjusting the pH of the suspension to physiological pH;
   d. optionally, isolating the composition, particularly by size exclusion/spin filtration.

The invention is further illustrated by the following examples and figures, from which further embodiments and advantages can be drawn. These examples are meant to illustrate the invention but not to limit its scope.

### DESCRIPTION OF THE FIGURES

FIG. 1 shows (A) Solvothermal synthesis of HfO₂ nanocrystals starting from 1 equivalent Hf(O-tBu)₄ and 88 equivalents benzyl alcohol. (B) (Diffusion filtered) 1H NMR spectra of MEEAA functionalized HfO₂ NCs in different solvents. The α and β resonances belong to the residual hydroxyl and methyl groups of methanol, respectively. (C) Transmission Electron Microscopy (TEM) images and data for the synthesized HfO₂ NCs. The NC diameter of the quasi-spherical NCs was calculated after measuring the surface area of at least 150 NCs and calculated the diameter as if it was a circle. Average: 2.64±0.19 nm. A size distribution histogram and a zoomed-in image of a singular NC can be seen respectively in the bottom left and the top right corner.

FIG. 2 shows (A) Ligand exchange performed between MEEAA functionalized NCs and PA-PEG. (B) 1H NMR reference spectra in MeOD of the free ligands as reference and the stepwise titration of MEEAA functionalized NCs with PA-PEG, equivalents are with respect to the total amount of MEEAA present. The resonance * is an unidentified impurity. (C) ³¹P NMR spectra (4096 scans) in MeOD for the stepwise titration of MEEAA functionalized NCs with PA-PEG, broadened signals are indicative of NC binding. (D) Diffusion filtered 1H NMR spectra of MEEAA functionalized NCs in MeOD after addition of 1.3 equivalents of PA-PEG. Signals arising from bound MEEAA are denoted in red, signals arising from PA-PEG are denoted in striped blue. C_{NC}=1210 μmol·L⁻¹, corresponding to 34 mg NCs of this size in 0.5 ml MeOD.

FIG. 3 shows a diffusion filtered 1H NMR spectrum of the NC suspension in MeOD at 1.6 equivalents PA-hex-PEG added.

FIG. 4 shows (A) and (B) ³¹P NMR spectra of PA-PEG and PA-hex-PEG functionalized NCs at different D₂O volume %. (C) Free ligand fraction for PA-PEG and PA-hex-PEG at different D₂O volume %, determined by peak deconvolution.

FIG. 5 shows (A) Ligand exchange performed between MEEAA functionalized NCs and nitrodopamine-mPEG. (B) 1H NMR spectra before and after the ligand exchange titration performed in D₂O with nitrodopamine-mPEG. 1.5 equivalents of nitrodopamine-mPEG were added and the pH was kept above 5 at all times during addition, the purified nitrodopamine functionalized NC spectrum was measured at pH=7.4. C_{NC}=128 μmol·L⁻¹, corresponding to 14.4 mg NCs of this size in 2 ml D₂O.

FIG. 6 shows effect of pH on ligand binding and stability in water for purified NCs functionalized with PA-PEG, PA-hex-PEG and Nitrodopamine-mPEG. (A) Bound and unbound ligand fraction in D₂O based on NMR peak deconvolution at different pH values. (B) Z-average value of NCs in DLS at different pH values. (C) Zeta potential of the NCs at different pH values. All measurements were performed at constant ionic strength (0.01 mol·L⁻¹ NaCl) at 25° C.

FIG. 7 shows stability of functionalized NCs in different concentrations of phosphate buffered saline (PBS) at pH 7.4 and 25° C. (A) Colloidal stability of functionalized nanocrystals measured using DLS z-average values at different PBS concentrations. (B) Stability of functionalized NCs in 2×PBS over time at pH 7.4 and 25° C.

FIG. 8 Colloidal stability map for metal oxide nanocrystals functionalized with carboxylic acids, phosphonic acids and catechols. In this concrete example, the inventors used HfO₂ as NC model system with its respective iso-electric point (IEP), MEEAA as carboxylic acid, PA-PEG as phosphonic acid and nitrodopamine-mPEG as catechol. A green color on the map correlates to good colloidal stability, the areas where a gradient transition to red is made shows reduced colloidal stability, red shaded areas indicate bad colloidal stability.

FIG. 9 shows UV-VIS spectra of purified nitrodopamine-mPEG functionalized NCs at different pH values in H₂O.

FIG. 10 shows the current clinical workflow for the detection of sentinel lymph nodes compared with the inventors' proposed workflow. Both are exemplified with a female breast cancer patient.

FIG. 11 shows (A) solvothermal synthesis of HfO₂ nanocrystals. (B) TEM image of the HfO₂ NCs. The major and minor diameter of the spheroid shaped NCs was determined by measuring at least 400 particles. A size distribution histogram can be seen in the INSERT corner. (C) 1H NMR spectrum of nitrodopamine-mPEG functionalized HfO₂ NCs in D₂O. (D) Volume size distribution of the HfO₂ NCs in PBS determined via Dynamic Light Scattering at 38° C. (E) HfO₂ NCs concentration series in PBS measured in CT, contrast enhancement was expressed in Hounsfield Units (HU).

FIG. 12 shows (A) Functionalization workflow to covalently link the dye to the NC surface. (B) Top: Excitation and emission spectra of IRDye 800CW-DBCO in PBS. The excitation spectrum was recorded while tracking emission at 805 nm. The emission spectrum was recorded while exciting at 730 nm. Bottom: Excitation and emission spectra of the NC-dye conjugate in PBS. The excitation spectrum was recorded while tracking emission at 813 nm. The emission spectrum was recorded while exciting at 764 nm.

FIG. 13 shows (A) coronal and sagittal slices of CT scans (slab thickness of 0.2 mm) taken at different timepoints of a mouse injected subcutaneously in the left hind footpad with an NC dose of 0.38 mg NCs/gram body weight. (B) Volume render of the 75 min post-injection scan. The SLN, skeleton and soft tissues have been segmented separately.

FIG. 14 shows volume renders of coregistered SPECT and CT scans taken at different timepoints of a mouse injected subcutaneously in the left hind footpad with a Nanocoll dose of 8.5 MBq.

FIG. 15 shows full structure of IRDye® 800CW DBCO.

### EXAMPLES

### Example 1: The HfO₂-MEEAA Model System

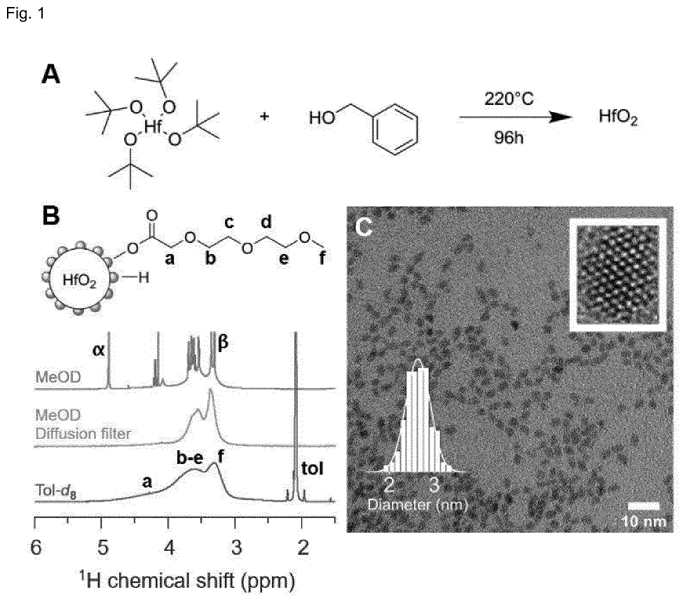

The inventors synthesized HfO₂ nanocrystals (NCs) from hafnium tert-butoxide and benzyl alcohol at 220° C. via an established solvothermal process (Lauria et al. ACS Nano 2013, 7 (8), 7041-7052) (FIG. 1A). The HfO₂ surface was functionalized with 2-[2-(2-methoxyethoxy)ethoxy]acetic acid (MEEAA, see FIG. 1B) to stabilize the nanocrystals in toluene and all unbound ligands were removed as described previously (De Roo et al. Chem Mater 2018, 30 (15), 5485-5492). The nanocrystals have a diameter of 2.64±0.19 nm (μ+σ) according to TEM (FIG. 1C) and possess the monoclinic crystal structure according to x-ray scattering analysis (data not shown). The 1H NMR spectrum of the nanocrystal dispersion in toluene-d₈ only shows broadened resonances, assigned to bound ligands (FIG. 1B). Indeed, spectral broadening is a typical attribute of bound ligands due to both homogeneous broadening (T₂ relaxation) and heterogeneous broadening (imperfect solvation of the ligand shell; see de Roo, ibid.).

The nanocrystals can also be dispersed in ethanol, methanol and water, and are thus an ideal starting point for the inventors' investigation into ligand binding behaviour in polar solvents. In methanol-d₄ (MeOD) the 1H NMR spectrum looks markedly different with sharp signals superimposed on the broad resonances (FIG. 1B). The inventors assign the sharp signals to auto-desorbed ligands, corroborated by the appearance of two sets of resonances in Diffusion Ordered Spectroscopy (DOSY). DOSY allows to separate (overlapping) NMR resonances according to their diffusion coefficient and thus separates the free ligand (quickly diffusing) from ligands bounds to NCs (slowly diffusing). The inventors can selectively observe the bound ligands in a diffusion filtered spectrum (FIG. 1B), and upon close inspection, one observes that the bound MEEAA resonances in methanol-d₄ are slightly sharper than the ones in toluene-de indicating a better solvation of the ligand shell in methanol. Given that all ligands were bound in toluene, the inventors infer that the solvent clearly plays a role in the adsorption-desorption equilibrium, for instance by changing the solubility of the ligand. Indeed, even more MEEAA ligands desorb in D₂O, but the nanocrystals remain stable in the pH range 2-6. The NCs quickly precipitate at pH>6 and therefore MEEAA is not a suitable ligand for many biomedical applications, which typically require stability at physiological conditions (pH=7.4).

Note that aside from the main MEEAA signals, the inventors also observed broad resonances with low intensity in the aromatic region of the 1H spectrum, assigned to benzoate ligands. Benzoic acid was previously identified as a side-product of the nanocrystal synthesis and found adsorbed on the nanocrystal surface. Functionalization of the surface with MEEAA after synthesis clearly did not remove all benzoate from the surface and a small fraction remains present.

### Example 2: HfO₂ Nanocrystal Synthesis and Functionalization

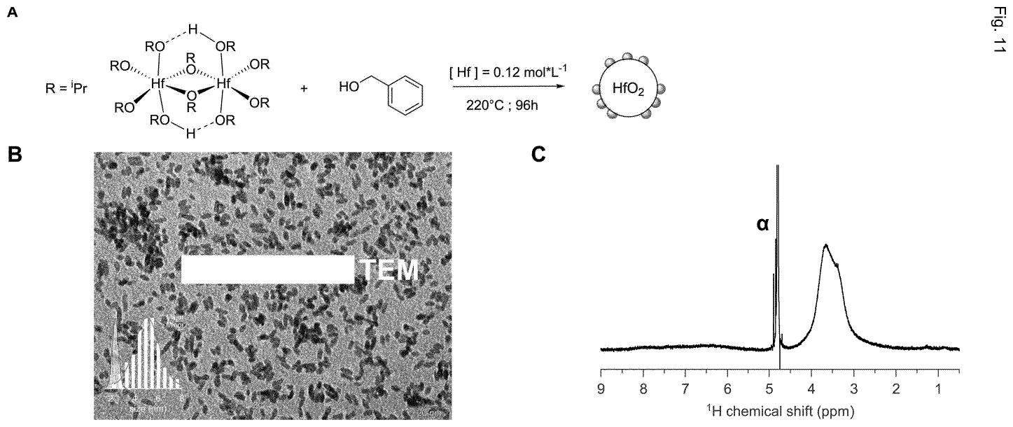

The inventors synthesized HfO₂ NCs from hafnium (IV) isopropoxide isopropanol adduct and benzyl alcohol at 220° C. by adapting an established solvothermal process (FIG. 11A). After synthesis, the NCs were functionalized with 2-[2-(2-methoxyethoxy)ethoxy]acetic acid (MEEAA) to stabilize them in toluene, all unbound ligands were removed as described previously. Importantly, to achieve comparable lymphatic drainage as the clinically used ^{99m}Tc labeled nanoparticles (i.e. Nanocoll) that have a mean diameter of around 8 nm, the inventors chose to synthesize HfO₂ NCs that are of the same size order after surface functionalization, whilst remaining below the renal clearance limit. The NCs have a major and minor diameter of respectively 5.04±3.73 and 2.41±0.85 nm (μ±3σ) according to transmission electron microscopy (TEM) (11B) and possess the monoclinic (P2₁/c) crystal structure according to X-ray Diffraction.

Nitrodopamine-mPEG was synthesized and purified as reported before, a ligand exchange was then performed on the MEEAA functionalized NCs in water and, after purification via spin-filtration (a form of ultrafiltration), a pure NC suspension is obtained. The 1H NMR spectrum of nitrodopamine-mPEG functionalized NCs (FIG. 11C) displays only broad peaks. Peak broadening is a typical attribute of bound ligands and is caused by both heterogenous line broadening (correlated to ligand shell solvation) and homogenous line broadening (T2 relaxation). The NC solution has a Z-average value of 12 nm in PBS according to dynamic light scattering (DLS) (FIG. 11D). The Z-average value is a single value that describes average particle size in solution and is highly sensitive to agglomeration, the match between the observed peak in DLS and this low Z-average value confirms the suspension to be free of any agglomeration. Thanks to the colloidal stability provided by nitrodopamine-mPEG, highly concentrated NC suspensions in PBS can be created, FIG. 11E shows a concentration series of functionalized NCs in PBS scanned using CT at a tube potential of 50 kV. A linear increase in X-ray attenuation with NC concentration can be observed, reaching over 6000 HU units, much higher than even the densest of bones.

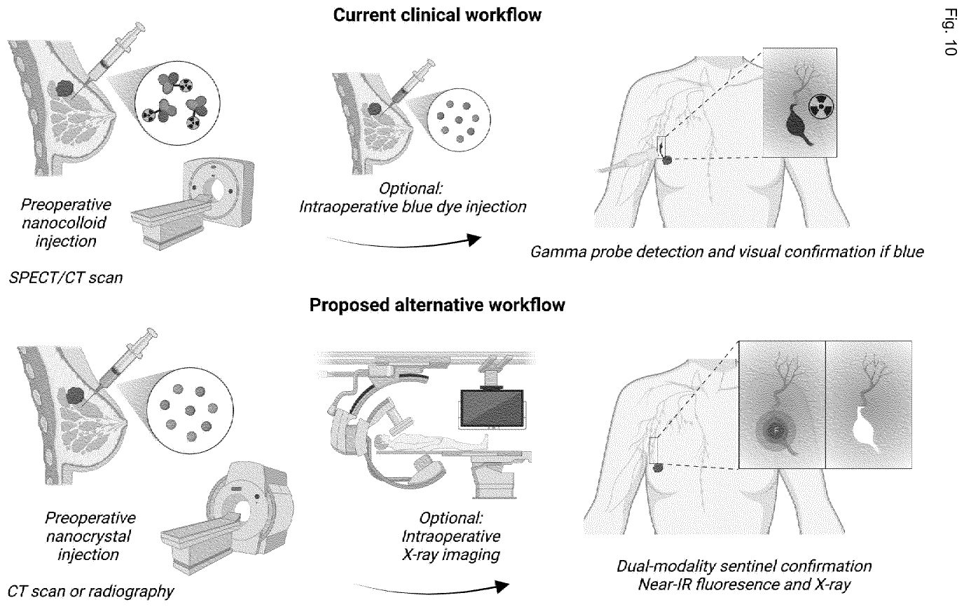

To provide a method to covalently link a payload to the NC surface, in the inventors' case dye molecules; the inventors synthesized 1-azido-N-(4,5-dihydroxy-2-nitrophenethyl)-3,6,9,12-tetraoxapentadecan-15-amide (nitrodopamine-PEG(4)-N₃), see FIG. 10. A one-step nitration reaction starting from dopamine hydrochloride results in the formation of nitrodopamine hemisulfate. The coupling between nitrodopamine hemisulfate and 15-Azido-4,7,10,13-tetraoxa-pentadecanoic acid succinimidyl ester (NHS-PEG(4)-N₃) was performed using N-methylmorpholine (NMM) acting as a non-nucleophilic base. The final nitrodopamine-PEG(4)-N₃ ligand was purified using preparative high-performance liquid chromatography (HPLC) and fully characterized with electrospray ionization/high-resolution mass spectrometry (ESI-HRMS) and NMR spectroscopy.

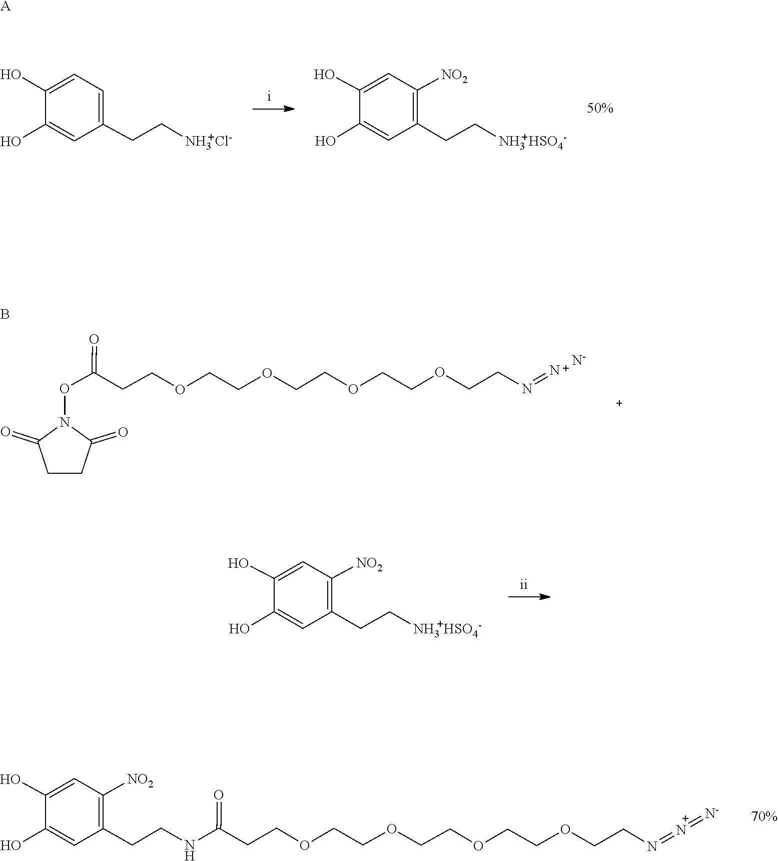

Scheme 1: (A) Synthesis of Nitrodopamine Hemisulfate (B) Synthesis of Nitrodopamine-PEG(4)-N₃. Reagents and conditions: (i) NaNO₂, 20% H₂SO₄, H₂O 0° C. to RT, 12 h, 50%; (ii) NMM, dry DMF, RT, 48 h, 70%.

The design of nitrodopamine-PEG(4)-N₃ was decided based on following criteria: The linker needs to be able to strongly bind to the NCs surface, in this case via the nitrocatechol anchor, while also containing a functional group that can covalently bind a payload after NC functionalization and purification. The inventors chose to introduce a terminal azide since this group can perform a fast bio-orthogonal copper-free click reaction with any molecule containing a cyclooctyne functionality. The PEG spacer was chosen to be slightly longer than nitrodopamine-mPEG to allow the azide functionality to reside outside the crowded ligand shell, reducing any possible steric hindrance that could occur during payload coupling while not significantly increasing the solvodynamic diameter of the NCs.

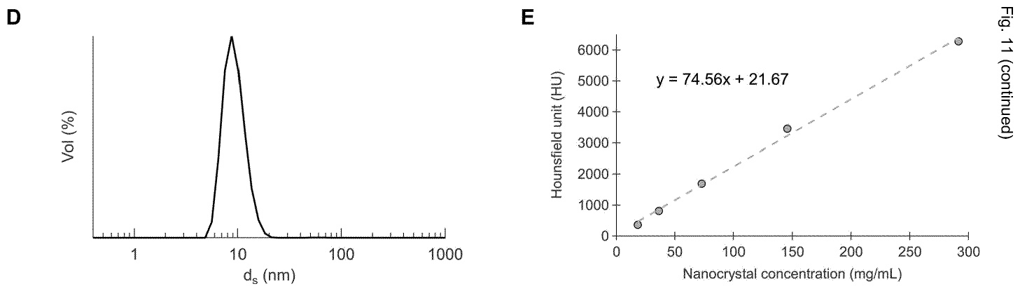

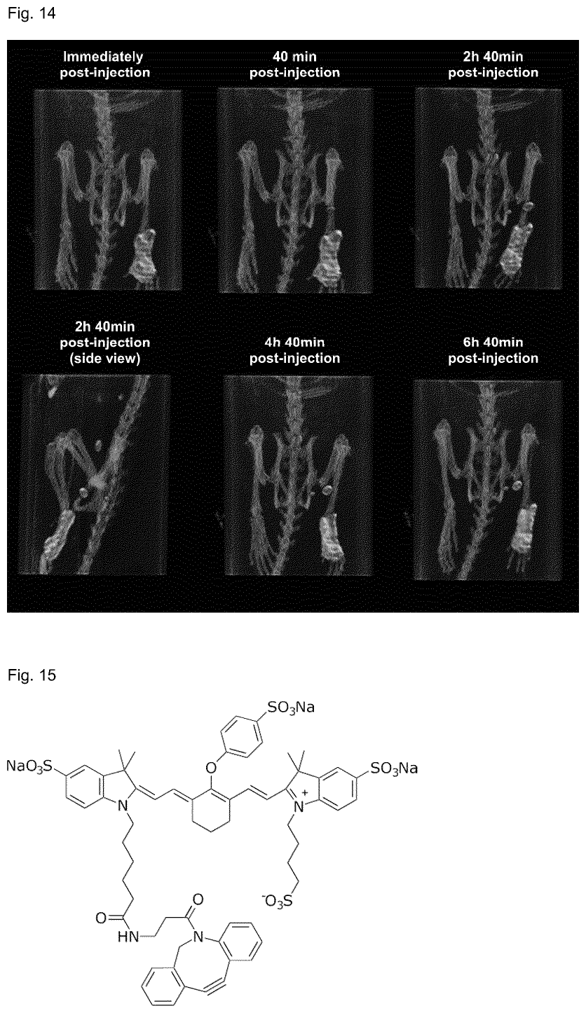

The inventors used the workflow depicted in FIG. 12A to perform dye coupling to the NCs. In a typical functionalization with nitrodopamine-mPEG, the inventors start from MEEAA-stabilized NCs in water and add 1.2 equiv deprotonated nitrodopamine-mPEG to perform the ex-change, after purification the bound organic mass on the NCs based on TGA and NMR is 18.9m %, resulting in a ligand density of 1.8 nm-2 and 60 ligands per NC, see SI for calculations. In the case of the mixed ligand shell, containing both nitrodopamine-mPEG and nitrodopamine-PEG(4)-N₃, the inventors aimed to have approximately one azide functionality per NC. With this goal in mind the inventors created a mixture of 1.18 equiv. nitrodopamine-mPEG and 0.02 equiv. nitrodopamine-PEG(4)-N₃, preactivated it, and performed the ligand exchange with MEEAA functionalized NCs in water. After spin-filtration only broad bound peaks can be observed via 1H NMR, indicating that purification was successful. Unfortunately, the broad nature of the peaks, and the spectral overlap between nitrodopamine-mPEG and nitrodopamine-PEG(4)-N₃ makes it impossible to quantify the azides bound to the NCs via ERETIC. However, the inventors can quantify this in an indirect manner through the next step in the workflow: dye coupling. The inventors chose the commercially available IRDye® 800CW DBCO as the inventors' dye of choice (see FIG. 15). This dye is a frequently used near-infrared (NIR) fluorescent tracer, currently used in multiple clinical trials for targeting malignancies in breast, pancreas, head and neck, and glioma. The dye both absorbs and emits light in the near-infrared spectrum, thus reducing spectral overlap with tissue autofluorescence. Moreover, it is compatible with (pre-)clinical grade imaging devices. 1 equiv (compared to the amount of azides that are assumed to be attached to the NCs) of IRDye® 800CW DBCO was dissolved in endotoxin-free ultrapure water at pH 7 and added to the azide-functionalized NCs, after stirring for 2 hours at 30° C. the suspension was purified again via spin-filtration using endotoxin-free water as solvent. By analyzing the sample concentrate and filtrate using UV-VIS spectroscopy and applying the Beert-Lambert law the inventors were able to quantify the bound and remaining unbound dye, thus indirectly measuring the dye-coupling efficiency. The inventors found that after coupling there are approximately 0.7 dye molecules covalently bound per NC. FIG. 12B shows normalized excitation and emission spectra of respectively freely diffusing dye molecules (top) and the NC-dye conjugate (bottom), both measured in PBS. While NIR-dyes typically show strong absorbance in the NIR region, this does not necessarily correlate to a strong excitation efficiency in said region. The inventors can see that while tracking emission at 805 nm for the free dye, the most efficient region to excite the molecule is actually in the visible region. Interestingly, the excitation spectrum of the NC-dye conjugate looks remark-ably different, while tracking emission at 813 nm the most efficient excitation region here is in the NIR. While the elucidation of the exact mechanisms for this phenomenon lie outside the scope of this work, the inventors could hypothesize that the absorption of nitrodopamine-mPEG in the 300-500 nm region influences the excitation of the conjugated dye. Using DLS the inventors observed that dye conjugation does not influence NC colloidal stability and does not cause a significant increase in hydrodynamic diameter compared to the mixed-shell functionalized.

### Example 3: Fluorescence Optimization of the NC-Dye Conjugate

Next, the inventors determined if concentration quenching occurs for the NC-dye conjugate's fluorescence emission. In order to exclude possible effects of nanocrystal concentration on fluorescence intensity, the inventors created a concentration series where the conjugated fluorescent dye concentration decreases, while retaining equal amounts of NCs in each sample. To achieve this the inventors mixed a suspension of dye-conjugated NCs with a suspension of nitrodopamine-mPEG functionalized NCs, and diluted with PBS accordingly for each sample. An in vivo imaging system was used to visualize sample fluorescence using band pass excitation filters at respectively 710 (±15) nm and 745 (±15) nm, while emission was observed in the ICG window (810-875 nm). A clear trend can be observed from the concentration series, wherein fluorescence intensity increases with increasing conjugated dye concentration, reaching an optimum at about 28 μmol*L⁻¹. Above this concentration, detrimental effects from quenching cause the fluorescence signal to decrease again. More specifically, the lowest conjugated dye concentration of 14.4 μmol*L⁻¹ exhibits approximately double the average radiant efficiency, a unit used to compensate for the non-uniform excitation light pattern, than the highest concentration of 460 μmol*L⁻¹. This trend and dye concentration optimum are found for both excitation wavelengths, with the sole difference being a higher average radiant efficiency for each sample with the 745 (+15) nm excitation filter (Table 1). As such, the 745 (+15) nm filter will be the optimal choice for further in vivo experiments. To showcase that each fluorescent sample contained equal amounts of NCs, CT scans were taken.

TABLE 1
Average Radiant Efficiency data extracted from the concentration
quenching experiments. A circular region-of-interest was drawn
for each sample, from which the Average Radiant Efficiency was
obtained with detection in the ICG window (810-875 nm).

| | 710 nm excitation: | 745 nm excitation: |
|---|---|---|
| Conjugated dye | Average | Average |
| concentration | Radiant Efficiency | Radiant Efficiency |
| (μmol/L) | [p/s/cm²/sr]/[μW/cm²] | [p/s/cm²/sr]/[μW/cm²] |
| 460 | 1.73E+09 | 2.20E+09 |
| 230 | 2.80E+09 | 3.70E+09 |
| 115 | 3.41E+09 | 4.30E+09 |
| 57.5 | 3.87E+09 | 4.93E+09 |
| 28.8 | 4.45E+09 | 5.89E+09 |
| 14.4 | 3.19E+09 | 4.52E+09 |

### Example 4: Preliminary In Vivo CT Optimisation

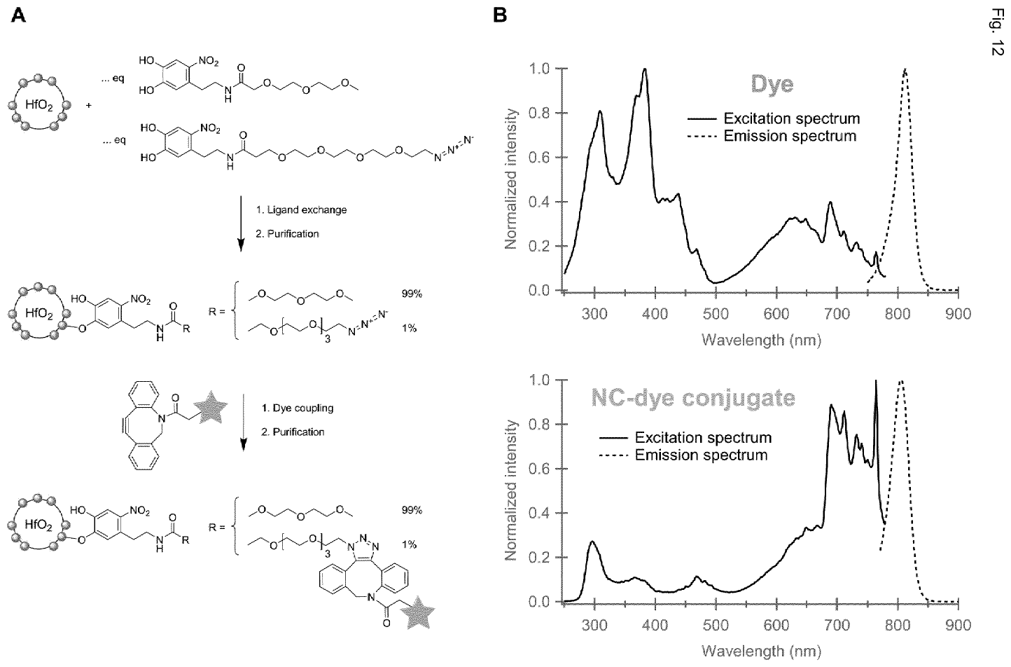

For mice, subcutaneous footpad injection is considered the standard injection route to achieve lymphatic drainage, with the popliteal lymph node, located behind the knee, as the SLN and the iliac, inguinal, sciatic and renal LNs as higher echelons. Anatomic regions of the LNs have been denoted on different CT slices, but the LNs themselves are difficult or impossible to distinguish from surrounding tissues. Starting from a NC suspension in PBS with a concentration of 291 mg HfO₂/ml, the inventors slowly injected 50 μL subcutaneously in the left hind foot of the mouse. 50 L is the maximum allowed, and feasible, injection volume in mice for this location and resulted in a dose of 0.38 mg NCs/gram body weight for this animal. Strikingly, immediately after injection strong contrast enhancement within the SLN (FIG. 13) can be observed, while no contrast enhancement in higher echelons is visible. Note as well that at this timepoint most of the contrast agent is still located in the footpad. 75 min post-injection, contrast enhancement remains strong within the SLN and a slight increase in contrast enhancement can be observed in the iliac LN, becoming stronger 150 min post-injection. The mouse was scanned longitudinally at several timepoints over a 24 h period, contrast enhancement within this period seems to remain stable in the SLN, the contrast enhancement in the iliac lymph node increases and stabilizes at approximately 4 h post-injection.

For each timepoint the inventors found that contrast enhancement is primarily located in the LN cortex, which is the entry point of lymph into the LN. The inventors hypothesized from this observation that the NCs are not retained for long in the medulla of the LN and quickly exit via efferent lymphatic vessels towards the next LN, and eventually into the bloodstream. Additionally, the inventors observed no ipsilateral to contralateral spillover from NCs, showcasing that the NCs follow a well-defined ipsilateral drainage route based on their injection location and do not leak prematurely from the lymphatic system. During the inventors' observations over time the animal showed no signs of pain or discomfort after NC injection and after initial recovery from the first sedation. However, in the interest of reducing drug dose and reducing total drainage time, the inventors tested two more dosages in different mice: 0.19 mg NCs/g bodyweight and 0.28 mg NCs/g bodyweight. From both doses the inventors observe similar drainage results as the highest 0.38 mg NCs/g bodyweight dose. Contrast enhancement for 0.19 mg NCs/g bodyweight is lower than the highest dose, and while contrast enhancement in the SLN is discernable directly after injection and could theoretically be used for SLN identification if the scan is timed right, a trained eye is required to distinguish it from the background for further scanpoints. The dose of 0.28 mg NCs/g bodyweight on the other hand does create strong contrast enhancement in the SLN for all scanpoints, while maintaining the required temporal separation in the appearance of NCs in higher echelons, as such this dose was chosen as a good compromise between increasing contrast enhancement while minimizing NC dose for following experiments. Furthermore, in the interest of future clinical applications it is also attractive to have contrast enhancement, and thus also NIR-fluorescence for the dual-modality probe, remain sufficiently high in the SLN for longer durations as surgical procedures can take several hours to complete. Additionally, depending on the SLN location of the treated malignancy, a radiograph could be taken as well instead of CT to confirm SLN location. During this initial CT optimization the inventors could notice the influence of slight anatomical variations between mice, as would be expected for any type of in vivo experiments using outbred animal strains. An example of this is the drainage behaviour of specific lymph nodes, while for the 0.28 mg NCs/g bodyweight the renal LN becomes visible at the 240 min scanpoint, the mouse having received a 0.38 mg NCs/g bodyweight dose does not show contrast enhancement in this LN at any timepoint. One should note as well that slight variations in anatomical locations of the specific LNs can be found between animals, for example the sciatic LN is generally small in size and located in close proximity to the pelvis, contrast enhancement from the NCs in this node can easily be mistaken for signal arising from the pelvis bone instead. Heed should be taken in this regard especially for mice where this anatomical closeness is even more pronounced.

### Example 5: Nanocrystal Formulation Approach

From the concentration quenching experiment (Table 1) and the preliminary in vivo CT experiments the inventors learned that there is a mismatch between the ideal dye concentration and the ideal NC concentration for CT contrast. As soft tissues inherently have a Hounsfield Unit value of around 100-300 in CT, a minimum amount of NCs present in the lymph nodes is needed to visualize them. On the other hand, soft tissues show (with a few exceptions) do not exhibit autofluorescence in the NIR and, in combination with the high sensitivity of the fluorescence imaging, very little amounts of dye are required. The concentration quenching experiments indicated that injection of an NC suspension where each NC contained one conjugated dye would be detrimental for the final fluorescence intensity, furthermore, other in vivo studies using NIR tracers have shown as well that as little as 1 μg of dye can be sufficient to visualize lymph nodes. 10 In order to satisfy the needs of both imaging modalities, instead of injecting an NC suspension where every NC contains a dye molecule, the inventors decided on a formulation approach where the majority of the NCs do not have a dye bound. As such, the formulation consists of a mixture of NCs functionalized solely with nitrodopamine-mPEG (see FIG. 11A) and NCs that have approximately 1 dye/NC conjugated (see FIG. 12), the injected NC dose would still be 0.28 mg NCs/g bodyweight but the conjugated dye concentration is only approximately 28 μmol*L-1. To ensure that both the NCs containing no dye and NCs that have a dye conjugated exhibit the same lymphatic drainage, a control experiment was performed. Within this control experiment a mouse was injected simultaneously in the right hind foodpad with a 0.28 mg/ml dose of NCs containing no dye and a 0.28 mg/ml dose of NCs in the left hind footpad containing approximately 1 dye/NC. The inventors could observe simultaneous contrast enhancement in both left and right SLNs after injection, becoming stronger over time. Accompanying this process, the left SLN showed clear signs of NIR fluorescence which could be detected through the skin, while the right SLN and foot expectedly did not. 180 min post-injection and beyond, a secondary NIR fluorescent signal appeared in the bladder, indicating the start of renal clearance of the NCs.

### Example 6: Comparison with ^{99m}Tc-Nanocoll

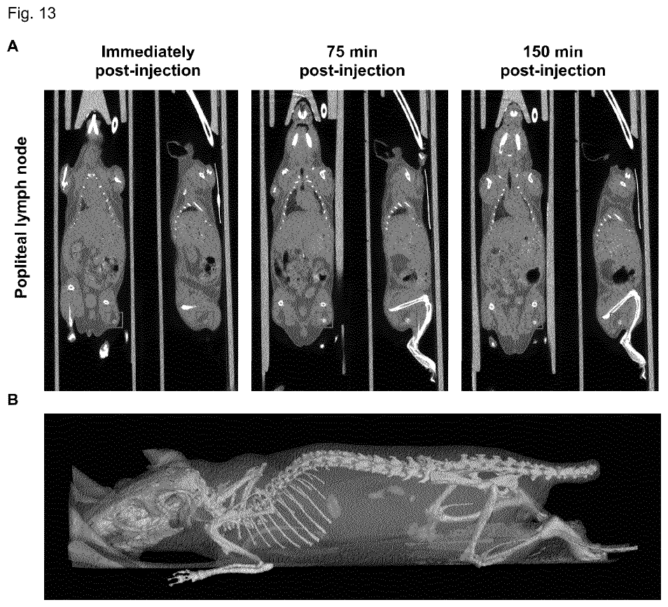

Finally, the inventors compare the results of the inventors' NCs to the current clinical standard, the inventors injected 3 mice subcutaneously in one of the hind footpads with 50 μL of 99m-Tc-Nanocoll, each injection having an activity between 6 and 9 MBq. Using a preclinical SPECT scanner, mice were scanned immediately after injection, and 40 min, 160 min, 280 min and 400 min post-injection. The chosen timepoints were less flexible than those for the NCs using CT due to the inherently longer acquisition times of SPECT. To obtain an acceptable signal-to-noise ratio the inventors reduced the field-of-view to an area covering the popliteal, iliac, sciatic, inguinal and renal LNs and scanned for 30 mins. The inventors chose not to scan beyond the 400 min as the half-life of the injected radioactive tracer is 6 hours. Volume renders for each scanpoint can be found in FIG. 14, important to note is that each SPECT scan has been coregistered with a CT scan, as it is difficult or impossible to obtain anatomical information from a SPECT scan.

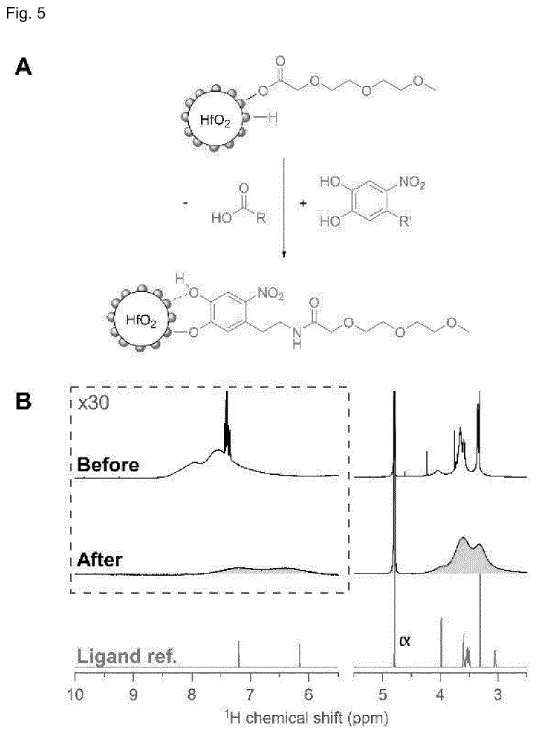

Similar to the NCs, the SLN becomes visible immediately after 99mTc-Nanocoll injection. The activity increases after 40 mins, but still only the popliteal LN can be discerned. After 160 mins not only the popliteal and iliac LNs, but also the sciatic and renal LNs were visible. Furthermore, as can be seen from FIG. 5 the sciatic LN is located in close proximity to the pelvis, which could make it challenging to distinguish LN contrast enhancement from the bone in that region. Similar to the NCs, 99mTc-Nanocoll activity in the LNs remains stable throughout the following 4 hours, and activity is mostly situated in the LNs cortex. After roughly 24 h most of the product has decayed.

### Example 7: Clinical Integration of Nanocrystals

FIG. 10 compares the current clinical workflow with how the inventors' the dual-modality NCs could be integrated using available infrastructure. After a singular preoperative injection of the NCs, a preoperative CT scan is taken. The non-radioactive nature of the NCs and the fast scan times of CT compared to SPECT/CT would create more flexibility concerning the timing of injection before surgery. Next, once the SLN has been identified via CT, intraoperative NIR-fluorescence is used to confirm SLN location and is used to guide their complete resection.

Nonetheless, it is possible that surgery will be performed several hours after initial injection, increasing the likelihood that higher echelons will be visible as well, as the inventors have shown for both the dual-modality NCs and 99mTc-Nanocoll. This should however not pose a problem as the preoperative scans are timed specifically to identify which node is the SLN, thus providing the surgeon with the required information to remove the correct nodes. Additionally, if available in the operating room, a C-arm could be used as a way to intraoperatively confirm SLN presence.

By removing radioactivity from the equation, the inventors could reduce overall patient radiation exposure and remove the need for high-cost SPECT scanners, instead using more widely available and faster techniques like CT or conventional radiography to identify SLNs. The second imaging modality, NIR-fluorescence, is part of the same imaging probe and as such does not suffer from undesired tissue extravasation like the currently used small molecule dyes do.

### Example 8: Competitive Binding of Phosphonic Acids

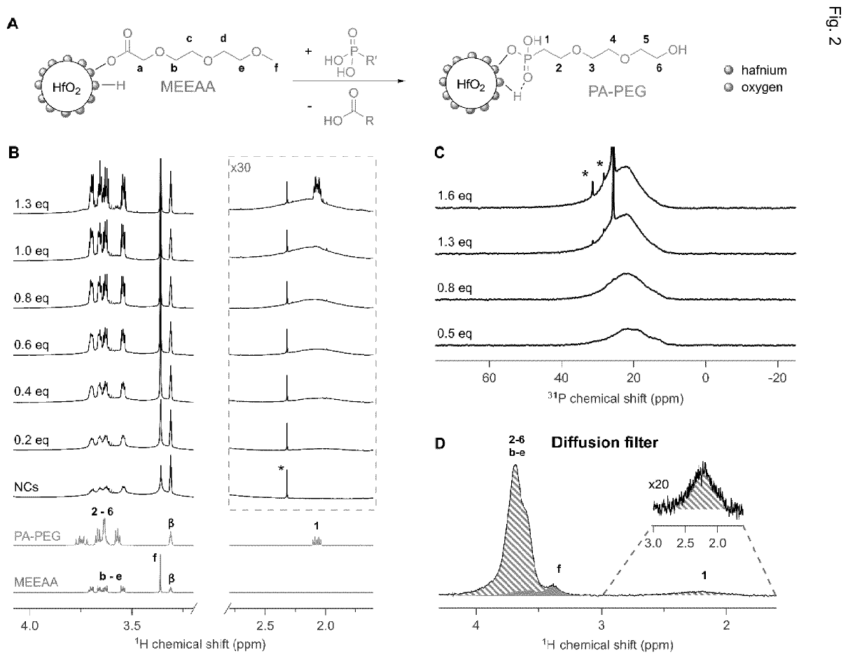

To evaluate the binding strength of phosphonic acids in methanol, the inventors chose (2-(2-(2-hydroxyethoxy)ethoxy)ethyl)phosphonic acid (PA-PEG) as a ligand with comparable structure to MEEAA, (FIG. 2A). In the ¹H NMR spectrum, resonance 1 of PA-PEG has a chemical shift of 2.05 ppm, clearly separated from the MEEAA resonances, allowing the inventors to selectively monitor the binding of PA-PEG (FIG. 2B). Since MEEAA has a methoxy group and PA-PEG has not, resonance f is used to gain selective information about the binding of MEEAA. The other resonances (2-6 and b-e) overlap.

Starting from MEEAA stabilized HfO₂ nanocrystals in methanol-d₄, the inventors add PA-PEG in steps while monitoring the ¹H NMR spectrum, see FIG. 1B. During the titration, the inventors observe the gradual appearance of a broad resonance around 2.2 ppm, assigned to resonance 1 of bound PA-PEG. Concomitantly, resonance f becomes more narrow (FIG. 2B), indicating the removal of MEEAA from the nanocrystal surface. Within the aromatic region the inventors also observe desorption of benzoic acid. The inventors conclude that PA-PEG is effectively displacing MEEAA and benzoic acid. Given the absence of free phosphonic acid, the exchange is quantitative for most of the titration. After addition of 1.3 equivalent of phosphonic acid, sharp signals appear in the same region, indicating that there is now free PA-PEG present. The same conclusions are drawn from the ³¹P spectra as well (FIG. 2C), where first a broad signal grows in intensity and a sharp ³¹P signal appears after adding more than one equivalent. Interestingly, the bound PA-PEG resonance still increases slightly in intensity between 1.3 and 1.6 equivalents. The inventors infer that the last part of the exchange does not proceed quantitatively and the remaining carboxylate ligands are more difficult to remove. This observation implies that the binding affinity (ΔG_{ads}) is not a single, fixed quantity for all the MEEAA ligands, but rather a distribution, as has been previously shown for CdSe nanocrystals.

Unfortunately, the NMR spectra feature a lot of spectral overlap, with superimposed signals of free and bound ligands. At the end of the titration, the resonances of free ligands dominate the spectrum. To gain more insight in the composition of the ligand shell at that point, the inventors turn to the diffusion filtered spectrum (FIG. 2D). While resonances 2-6 overlap with resonances b-e, the clear presence of resonance f indicates the presence of residual MEEAA on the surface. Based on the diffusion filtered profile of bound MEEAA, the inventors assigned the part of the spectrum belonging to MEEAA as the red shaded area. The blue patterned area is assigned to PA-PEG. The ratio of the areas gives the inventors a rough estimate of the ligand shell composition: 10% MEEAA and 90% PA-PEG, but this estimation ignores differences in relaxation behaviour of the different resonances. There is also still benzoate present on the surface according to the regular ¹H spectrum but the signal-to-noise of these resonances was too low in the diffusion filtered spectrum due to their faster T₂ relaxation (because of rigidity and proximity to the surface). The above results thus confirm that the exchange between the carboxylate ligands and PA-PEG does not go to completion in methanol. This conclusion stands in contrast to the relative binding affinities of fatty acids and alkylphosphonic acids in nonpolar solvents, were phosphonic acids quantitatively replace carboxylic acids in a 1:1 stoichiometry.

The auto-desorption of MEEAA in methanol already indicates that the ligand solubility can change the binding affinity. Indeed, ligand binding is an equilibrium process, governed by the chemical potential of each species:

$$NC \text{(*} ) + L ⇌ NC ( L ) \qquad Eq . 1$$

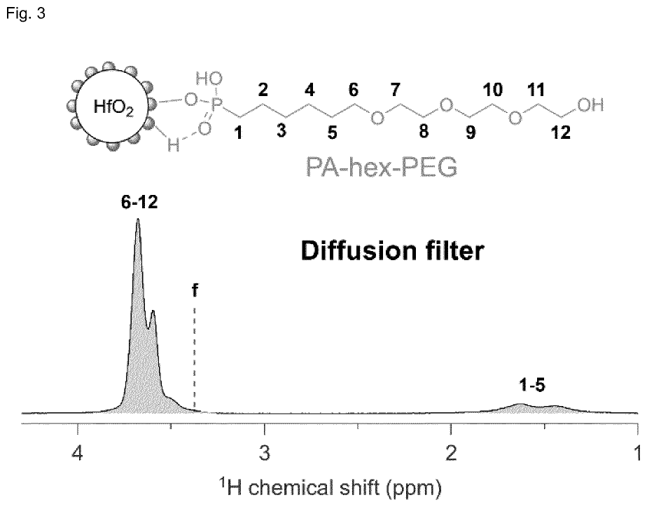

Therefore, this adsorption-desorption equilibrium is dependent on the chemical potential of the free ligand (and thus the solubility). To explore this concept in practice, the inventors designed a ligand shell architecture that would mimic a micelle; having both a hydrophobic and a hydrophilic segment. To this end the inventors chose the ligand (6-{2-[2-(2-hydroxy-ethoxy)-ethoxy]-ethoxy}-hexyl)phosphonic acid (PA-hex-PEG), see FIG. 3. The inventors hypothesized that this ligand would more readily self-assemble on the nanocrystal surface since the hydrophobic region decreases the solubility in polar solvents. Using PA-hex-PEG, the inventors performed the same titration experiment as before. The results are generally quite similar showing the exchange of carboxylate ligands for PA-hex-PEG (not shown). However, this time, most benzoate ligands are removed by PA-hex-PEG (although not completely) and hardly any MEEAA resonances are detectable in the diffusion filtered spectrum (FIG. 3). At most 3% of MEEAA is still present in the ligand shell. The inventors conclude that indeed; the competitive binding can be manipulated by changing the ligand solubility. Interestingly, benzoic acid appears to have a higher binding affinity than MEEAA which could be ascribed to both its higher acidity and its lower solubility in methanol.

### Example 9: Purification and Transfer to Water of Phosphonate Capped Nanocrystals

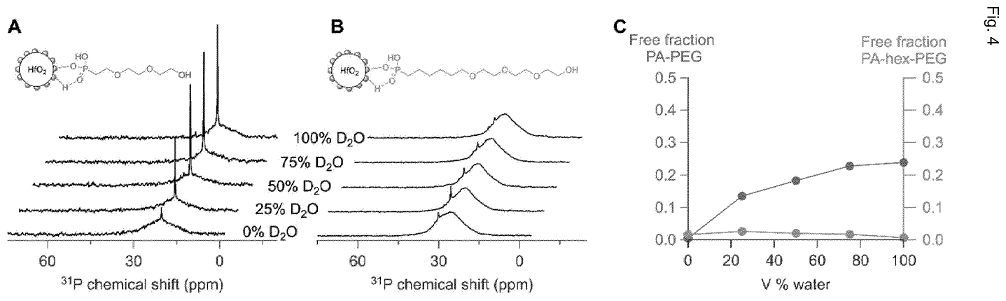

With the inventors' final goal in mind of providing a stable surface chemistry for biomedical applications, the inventors sought to purify the inventors' dispersions and disperse them in aqueous media. Precipitation-redispersion cycles are the most common way of purifying nanocrystals. However, the great versatility of the ethylene glycol segment provides colloidal stability in a broad range of solvents and non-solvents like hexane do not mix well with methanol. Therefore, the inventors choose to purify the inventors' dispersion using spin filtration, a form of ultrafiltration. The technique is based on semipermeable membranes (like dialysis) where small molecules can pass through the pores but large nanocrystals cannot. By performing the separation in a centrifuge, purification is expedited. First the nanocrystal suspension is placed in the spin filter and further diluted with pure methanol. Dilution does not induce ligand desorption. After filtration, a concentrated dispersion of nanocrystals is retrieved. The purification is successful as shown by the removal of almost all unbound species after 3 purification cycles. A comparison of the diffusion filtered spectra before and after spin filtration show a perfect match, proving that the purification did not change the ligand shell composition. Interesting differences between PA-PEG and PA-hex-PEG were observed when gradually changing the solvent composition from pure methanol-d₄ to D₂O (FIG. 4). Whereas PA-PEG gradually desorbs from the surface when increasing water content, PA-hex-PEG remains tightly bound. This further underscores the inventors' hypothesis that PA-hex-PEG behaves as a micelle mimic, avoiding contact of the hydrophobic segment with water. By binding the nanocrystal surface the PA-hex-PEG ligand creates a hydrophobic inner shell with alkyl-alkyl interactions and a hydrophilic outer shell with ethylene glycol moieties as hydrogen bond acceptor for water molecules. PA-PEG on the other hand is highly water soluble and thus the affinity for the hafnium oxide surface decreases upon increasing the water content.

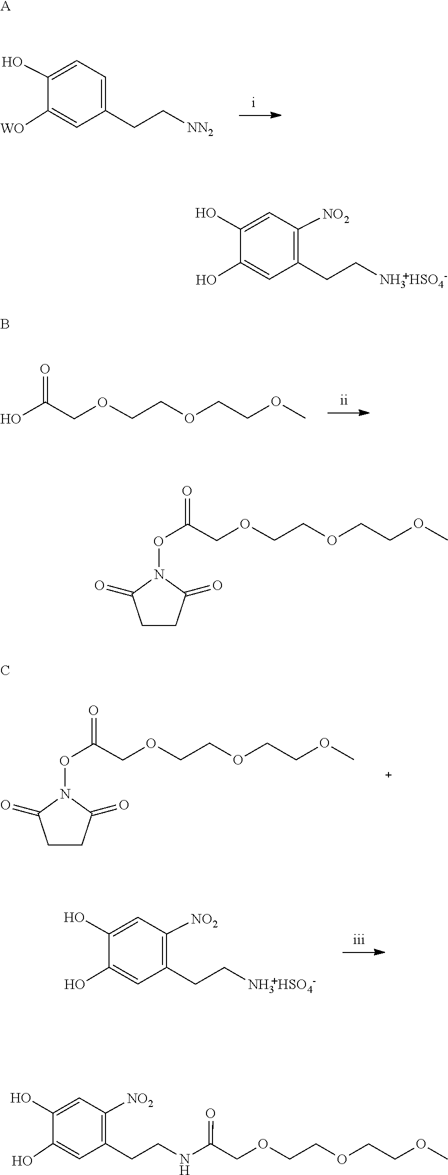

Scheme 2. (A) Synthesis of nitrodopamine hemisulfate. (B) Synthesis of MEEAA-NHS. (C) Synthesis of nitrodopamine-mPEG. Reagents and conditions: (i) NaNO₂, 20% H₂SO₄, H₂O, 0° C. to RT, 12 h, 50%; (ii) NHS, DCC, DMAP, dry THF, 0° C. to RT, 12 h, 83%; (iii) NMM, dry DMF, RT, 48 h, 75%.

### Example 10: Synthesis and Binding of Catechols

The inventors synthesized N-(4,5-dihydroxy-2-nitrophenethyl)-2-(2-(2-methoxyethoxy) ethoxy) acetamide (nitrodopamine-mPEG), see Scheme 2. The inventors opted for a nitrocatechol instead of an unsubstituted catechol since the nitro group decreases the catechol pKa values, and improves the oxidative stability of the catechol. Starting from dopamine hydrochloride, a one-step nitration reaction results in the formation of nitrodopamine hemisulfate. Separately, MEEAA was converted in an activated N-hydroxysuccinimide ester (MEEAA-NHS) using N,N′-dicyclohexylcarbodi-imide (DCC), N-hydroxysuccinimide (NHS) and 4-Dimethylaminopyridine (DMAP). The coupling between nitrodopamine hemisulfate and MEEAA-NHS was performed using N-Methylmorpholine (NMM) acting as non-nucleophilic base. The final nitrodopamine-mPEG ligand was purified using preparative HPCL and fully characterized with ESI-HRMS and NMR spectroscopy.

When the inventors performed a similar competitive binding experiment as before (addition of nitrodopamine-mPEG to MEEAA capped HfO₂ nanocrystals in methanol-d₄), the inventors found that one equivalent of nitrodopamine-mPEG was unable to effectively compete for the nanocrystal surface, even at merely 0.4 equivalents of catechol, freely diffusing nitrodopamine-mPEG signals could be observed. Such a low binding affinity was highly unexpected, given many reports of successful surface functionalization with catechols (Okada et al. ChemistrySelect 2018, 3 (29), 8458-8461; Dragoman et al. Chemistry of Materials 2017, 29 (21), 9416-9428; Amstad et al. The Journal of Physical Chemistry C 2011, 115 (3), 683-691; Amstad et al., Nano Letters 2009, 9 (12), 4042-4048; Gillich et al. Journal of the American Chemical Society 2011, 133 (28), 10940-10950; Xie et al. Adv. Mater. 2007, 19 (20), 3163-3166.; Bae et al. Bioconjugate Chemistry 2010, 21 (3), 505-512.). However, in these reports water or biological buffers were used as the solvent. Therefore, the inventors designed a competitive binding experiment directly in water. Fortunately, MEEAA stabilized HfO₂ nanocrystals are stable in D₂O, even though a significant portion of MEEAA is desorbed. The addition of one equivalent nitrodopamine-mPEG, without adjusting the pH, results in a turbid suspension with pH=2. When the pH is adjusted to pH=5, a stable suspension is obtained. To allow for a systematic, gradual addition of nitrodopamine-mPEG the inventors prepared a stock solution of nitrodopamine-mPEG with 2 equivalents of NaOD to doubly deprotonate the catechol. Addition of the base changes the color of the solution from light yellow to a deep burgundy. The inventors added this stock solution to a suspension of MEEAA stabilized HfO₂, in steps of 0.5 equivalents and recorded both standard 1H NMR and diffusion filtered NMR spectra. After addition of 0.5 equivalents, the inventors do not observe any sharp signals belonging to nitrodopamine-mPEG, while the inventors do observe desorbed benzoic acid and desorbed MEEAA. In the diffusion filtered spectrum, the inventors also see a clear change. In the aromatic region, the broad signals of benzoate are replaced by the broad signals of nitrodopamine-mPEG. The peak shape of the region 3-4 ppm is also altered. The exchange continues as more nitrodopamine-mPEG is added and upon addition of 1.5 equivalents, sharp (unbound) nitrodopamine-mPEG signals are detected. The suspension remains stable despite the pH being 10.3 at this point in the titration, providing further evidence that the ligand exchange was successful since MEEAA stabilized nanocrystals would precipitate at pH>6. The inventors conclude that nitrodopamine-mPEG can quantitatively displace MEEAA from the nanocrystal surface if the pH>5.

The NCs were again purified using multiple cycles of spin filtration, using Milli-Q water as solvent, until the filtrate was nearly colorless. The concentrate was evaporated and redispersed in D₂O, and the pH was adjusted to 7.4. FIG. 5B contains a regular ¹H NMR spectrum of the purified suspension, the purity of the sample is striking and only broadened resonances pertaining to nitrodopamine-mPEG are observed. Based on quantitative ¹H NMR and a TGA mass loss of 25.79% the inventors calculate a nitrodopamine-mPEG ligand density of 1.53 nm⁻² at the nanocrystal surface. The inventors conclude that nitrodopamine-mPEG forms a tightly bound ligand shell on the nanocrystals at physiological pH with no signs of desorption.

### Example 11: pH-Dependence of Ligand Binding

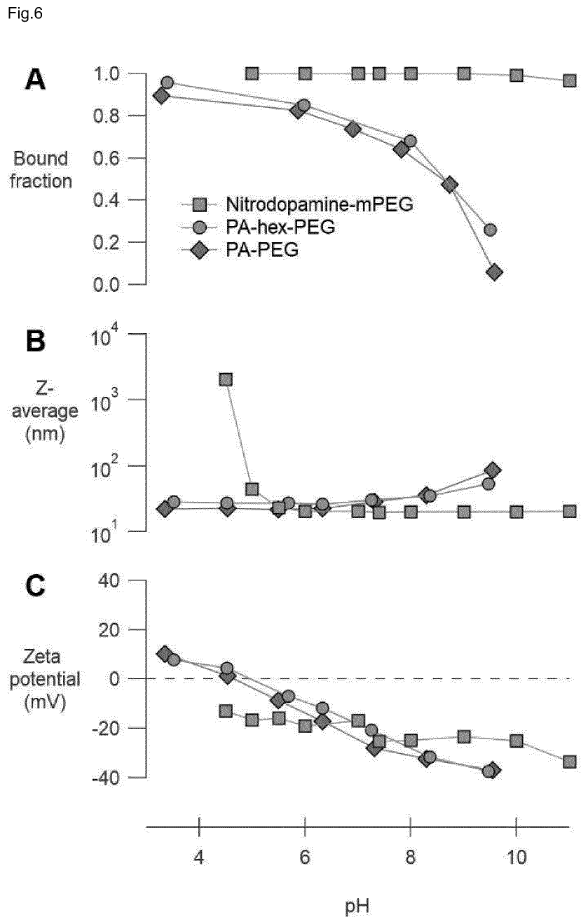

Since it is obvious that pH plays a crucial role in ligand binding in aqueous environments, the inventors systematically varied the pH from 3 to 10 and used ¹H NMR, ³¹P NMR and Dynamic Light Scattering (DLS) to assess the ligand binding and colloidal stability (FIG. 6). From NMR, the inventors extract the bound ligand fraction for the phosphonic acid ligands via peak deconvolution of the ³¹P resonances and for nitrodopamine-mPEG via peak deconvolution of the aromatic ¹H resonances. From DLS measurements the inventors obtained the Z-average value and the zeta potential. The Z-average value is a single value describing the average particle size and is most sensitive to agglomeration. The zeta potential indicates the degree of electrostatic repulsion between the nanocrystals (zeta potential values above +25 mV or below −25 mV indicate stable suspensions). From FIG. 6, the inventors clearly observe that the bound ligand fraction decreases with rising pH for both PA-PEG and PA-hex-PEG. Taking the pKa values of ethyl phosphonic acid (pKa₁=2.43; pKa₂=8.05) as reference, it is striking that the bound ligand fraction decreases most steeply around pKa₂. The inventors infer that the second deprotonation of the phosphonic acid causes ligand-ligand repulsion in the ligand shell and increases the solubility of the ligand in water. Both effects lead to a reduced bound ligand fraction. Not surprisingly, the loss of ligands has a detrimental effect on the colloidal stability and the Z-average increases significantly for pH>pKa2. Overall, PA-hex-PEG performs slightly better than PA-PEG but the difference is small. Most likely the double anionic phosphonate compensates for the short hydrophobic segment.

One might expect complete loss of colloidal stability upon ligand desorption. However, no visual turbidity was observed in the samples and the Z-average remains below 100 nm. Note that the zeta potential drops below −25 mV at pH>8. While steric stabilization is lost with progressing ligand desorption, electrostatic stabilization takes over, preventing the NCs from fully destabilizing. The negative charge could originate from residual bound, double deprotonated phosphonates, or more likely from hydroxide adsorption on the nanocrystal surface. Indeed, in water, there are multiple adsorption-desorption equilibria present simultaneously.

$$NC \text{(*} ) + {L}^{-} ⇌ {NC ( L )}^{-} \qquad Eq . 2$$

$$NC \text{(*} ) + {H}^{+} ⇌ {NC ( H )}^{+} \qquad Eq . 3$$

$$NC \text{(*} ) + {OH}^{-} ⇌ {( OH )}^{-} \qquad Eq . 4$$

Add also the acid-base equilibria and one starts to appreciate the complex pH dependence of the system.

According to FIG. 6, the phosphonic acids keep the nanocrystals stable between pH 3 and 8 in a static system. However, in a dynamic biological environment (e.g. a blood vessel), many competing ligands are present and desorbed ligands are quickly removed. The equilibrium will adjust and thus ligands will continuously desorb from the surface, eventually causing loss of colloidal stability. Despite an acceptable Z-average value at physiological pH, this dynamic behaviour could explain why single phosphonate ligands are not necessarily successful at fully preventing aggregation in physiological media.

Interestingly, nitrodopamine-mPEG has almost the complete opposite behaviour, unable to provide stable colloidal dispersions under acidic conditions with a very sharp transition around pH=5. This is evidenced by the steep increase in the Z-average (FIG. 6). Between pH 5 and 10, all nitrodopamine-mPEG ligands remain bound and only at pH=11, there is a slight decrease in the bound fraction from 100% to 97%. This translates in an excellent colloidal stability in pH range 5-11. The inventors conclude that for aqueous applications at physiological pH, nitrodopamine-mPEG outperforms both PA-PEG and PA-hex-PEG. For aqueous applications at acidic pH on the other hand the phosphonic acid ligands are better suited. To test if there would also be a temperature dependence on the binding dynamics of the functionalized NC systems, the inventors performed variable temperature NMR measurements in D₂O at pH 7.4 between 25° C. and 60° C. 1H NMR spectra showed no change in ligand adsorption/desorption equilibria for both phosphonic acids and nitrodopamine-mPEG, leading the inventors to tentatively conclude that the results from the pH titrations would also translate to physiological temperature.

To illustrate the complementary behaviour of phosphonic acids and catechols, the inventors performed a competitive exchange reaction on purified nitrodopamine-mPEG NCs in D₂O. 1 eq of PA-PEG was added, compared to the original amount added to functionalize the NCs with nitrodopamine-mPEG, and NMR measurements were performed at different pH values. The results clearly show that at acidic pH values a partial exchange with PA-PEG occurs, evidenced by the appearance of sharp nitrodopamine-mPEG signals in the aromatic region and methylene triplet around 3 ppm. The nitrodopamine-mPEG NC suspension, which normally would fully destabilize below pH 5, remained colloidally stable at pH 2.22 due to the now mixed catechol-phosphonate ligand shell. The exchange equilibrium mostly shifts back towards nitrodopamine-mPEG as the pH moves towards neutral and basic values.

### Example 12: Stability in Phosphate Buffered Saline (PBS)

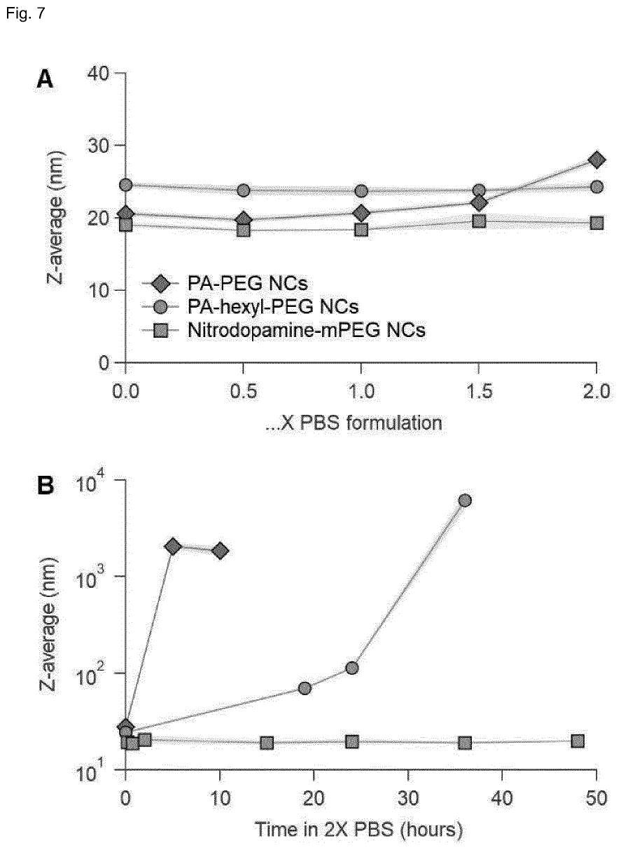

Finally, the inventors assessed the stability of NCs functionalized with PA-PEG, PA-hex-PEG and nitrodopamine-mPEG in phosphate buffered saline (PBS). Stability in PBS is an important prerequisite for biomedical applications, since many in vivo experiments inject the desired drug or contrast agent in saline solution or in PBS. A 1×PBS buffer contains 137 mmol L⁻¹ NaCl, 2.7 mmol·L⁻¹ KCl, 10 mmol·L⁻¹ Na₂HPO₄ and 1.8 mmol·L⁻¹ KH₂PO₄, and is the standard concentration. When the concentrations are half or double, these buffers are referred to as 0.5×PBS and 2×PBS respectively. It is clear that PBS contains relatively high salt concentrations and phosphate ions (which will compete for the surface) and thus provides a real test for the stability of the inventors' functionalized nanocrystals. First, the inventors varied the PBS concentration and immediately measured the Z-average via DLS (FIG. 7A). Up to 1.5× PBS, all ligands keep the nanocrystals colloidally stable. As expected, PA-PEG is the weakest ligand and cannot prevent the start of nanocrystal aggregation in 2×PBS solutions. Second, the stability of all functionalized NCs was monitored over time in 2×PBS (FIG. 7B). Clearly, PA-PEG functionalized nanocrystals agglomerate quickly and visually precipitate after several hours. The colloidal stability of PA-hex-PEG functionalized nanocrystals steadily decreases over 24 hours, before completely agglomerating. Only nitrodopamine-mPEG functionalized nanocrystals remain completely stable. There is no sign of aggregation over the course of 48 hours and the suspension also remains visually clear for at least 1 month.

### Example 13: Discussion

The above results make clear that surface chemistry become much more complex when moving from nonpolar to polar (e.g., aqueous) solvents. In nonpolar solvents, charged ligands or nanocrystals are thermodynamically unstable, leading to a limited set of binding motifs and clear ligand exchange rules. For example, auto-desorption of oleate (deprotonated oleic acid) does not occur in toluene. For the binding motif of PbS(PbX₂), removal of the entire Lewis acid PbX₂ has been observed in chloroform or coordinating solvents like THF. Likewise, for HfO₂(H,OOCR), desorption of oleic acid is possible by recombination of the carboxylate and proton. However, these restrictions disappear in polar solvents where charges are stabilized, and the proton and carboxylate have independent adsorption/desorption equilibria, see also Equations 2-4.

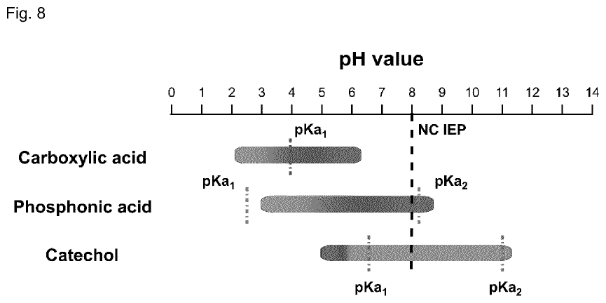

From the above data, the inventors constructed a colloidal stability map, indicating which ligands provide colloidal stability in specific pH ranges, see FIG. 8. It is interesting to correlate this stability map to the pKa's of the ligands. The inventors take the pKa values of ethyl phosphonic acid (pKa₁=2.43; pKa₂=8.05) as reference for the PA-PEG and PA-hex-PEG ligands. At pH=3 (where the particles are stable), the inventors calculate that the 96% of the phosphonic acid is mono-deprotonated. One can assume that the phosphonate will bind in this form to the nanocrystal surface. At pH>8 (above pKa₂), the bound ligand fraction decreases quickly and the particles start to aggregate (FIG. 6). The double deprotonation has two consequences; (1) the ligand becomes more soluble in water, and (2) there appears electrostatic ligand-ligand repulsion, instead of the stabilizing hydrogen bonding. Both effects promote ligand desorption. In addition, the iso-electric point of hafnia occurs at pH=8, rendering the surface negatively charged at pH>8, thus further decreasing the binding affinity of the negatively charged phosphonate ligand.

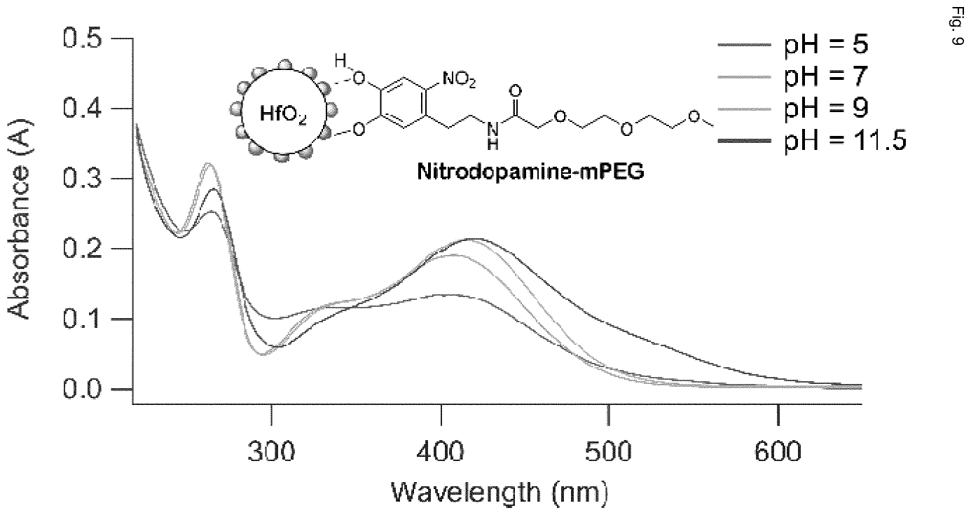

A similar reasoning applies to the case of nitrodopamine-mPEG (pKa₁=6.6; pKa₂=11; based on nitrodopamine pKa values). At pH=5 (where the particles are unstable), the inventors calculate that approximately 98% of the ligand is fully protonated, and can thus only interact with the surface via weak hydrogen bonds. Above pH 5, more nitrodopamine-mPEG becomes mono-deprotonated and is able to coordinate to the surface metal sites and provides an additional hydrogen bond to further stabilize the bound state. This is confirmed by comparing the UV-VIS spectra of nitrodopamine-mPEG bound to HfO₂ NCs with the reference spectra of the free ligand, see FIG. 9. For 5<pH<11, the inventors find the mono-deprotonated species. At pH>11, the inventors see again the appearance of free ligands in NMR and the double deprotonated species in UV-Vis. For the reasons mentioned above, the double deprotonated species has a low binding affinity for the surface. Finally, carboxylic acids can only be mono-deprotonated and the inventors observe that MEEAA (pKa=3.4) keeps the NCs stable until pH=6. The inventors hypothesize that they are less stable than phosphonates and catechols due to the lack of ligand-ligand hydrogen bonding.

The stability experiments in buffer solution point to two additional variables: competitive ligands and salt. In phosphate buffered saline, a high concentration of phosphate is present, which competes for the surface but does not provide steric stabilization. Since the phosphonic acids (PA-PEG and PA-hex-PEG) desorb at pH=7.4 (25% desorbed, see FIG. 6), they are in equilibrium and can thus be slowly displaced over time. The nitrodopamine-mPEG is tightly bound (0% desorbed) and is not being displaced by phosphonic acid, judging from the inventors' competition experiments. Hence the high stability of the particles capped with nitrodopamine-mPEG.

The above discussion shows that in aqueous media, surface chemistry is a complex interplay between multiple factors. There is the pH-dependent charge of the surface, the pH-dependent deprotonation of the ligand, and competition by phosphate in buffer solution. Having thus the pKa(s) of the ligands available and the isoelectric point of the nanocrystals, one can now use the colloidal stability map (FIG. 8) to start predicting which ligands will provide colloidal stability at a particular pH.

### Example 14: Synthetic Details

(6-{2-[2-(2-Hydroxy-ethoxy)-ethoxy]-ethoxy}-hexyl)phosphonic acid and (2-(2-(2-hydroxyethoxy)ethoxy)ethyl)phosphonic acid were purchased from SiKÉMIA. N-Hydroxysuccinimide (≥98%) and dopamine hydrochloride (≥99%) were purchased from Acros Organics. 2-[2-(2-Methoxyethoxy)ethoxy]acetic acid (≥95.0%) was purchased from TCI Chemicals. Hafnium (IV) isopropoxide isopropanol adduct (99.99%) was purchased from Fisher Scientific. IRDye 800CW-DBCO was purchased from LiCor. Hafnium (IV) tert-butoxide (99.99%), N,N′-Dicyclohexylcarbodiimide (99%), 4-(Dimethylamino)pyridine (≥99%), 4-Methylmorpholine (99%), Sodium nitrite (≥99.0%) and solvents used for synthesis were purchased from Sigma Aldrich. All purchased chemicals were used without further purification. All deuterated solvents were purchased from Sigma Aldrich or Eurisotop.

### Synthesis of MEEAA-NHS.

4 mmol (0.7128 g) 2-[2-(2-methoxyethoxy)ethoxy]acetic acid and 4.2 mmol (0.4828 g) N-hydroxysuccinimide were dissolved in 8 ml dry THF in a predried vial and cooled to 0° C. 4.2 mmol (0.866 g) N,N′-Dicyclohexylcarbodiimide was dissolved in a separate pre-dried vial in 4 ml THF and added dropwise to the first mixture by performing an air-free transfer. The mixture was stirred for 15 mins at 0° C., after which 0.2 mmol (0.024 g) catalytic 4-dimethylaminopyridine was added. The mixture was stirred overnight at room temperature, resulting in a white turbid mixture. The turbid solution was transferred to a 50 ml centrifuge tube and centrifuged 5 mins at 5000 rcf, the supernatant was transferred to a flask using a 0.2 UM PTFE syringe filter and the white solid was washed once with 10 ml THF to collect remaining product. After solvent removal using a rotary evaporator the viscous liquid was dissolved in 12 ml DCM, the organic phase was extracted four times with MQ water and two more times with brine. The organic phase was dried over MgSO₄ and dried using a rotary evaporator. Product was collected as a colorless viscous liquid with 83% yield.

¹H NMR (500 MHZ, CDCl₃): δ 4.5 (s, 2H) δ 3.8-3.76 (m, 2H) δ 3.7-3.66 (m, 2H) δ 3.65-3.6 (m, 2H) δ 3.55-3.51 (m, 2H) δ 3.36 (s, 3H) δ 2.82 (s, 4H). ¹³C NMR (500 MHz, CDCl₃): δ 168.73 (s) δ 166.02 (s) δ 77.24 (s) δ 71.91 (s) δ 71.37 (s) δ 70.607 (s) δ 70.602 (s) δ 66.55 (s) δ 59.06 (s) δ 25.58 (s). HRMS 275.26 calc for [M], 292.9 [M+NH₄]⁺ found.

### Synthesis of Nitrodopamine Hemisulfate.

8.753 mmol (1.66 g) dopamine hydrochloride and 35.219 mmol (2.43 g) NaNO₂ were dissolved in 100 ml MQ water and cooled in an ice bath. 8.33 ml of precooled 20% H₂SO₄ was added dropwise to the mixture under heavy stirring, during addition the mixture turns turbid yellow with the formation of brown gasses. The mixture was removed from the ice bath and allowed to stir 12 h at room temperature. The resulting turbid yellow solution was cooled again in an ice bath, followed by collection of the solid via suction filtration using a por 4 fritted glass filter. Next, the solid was washed 2 times with 50 ml ice cold MQ water, 1 time with 50 ml ice cold absolute ethanol and 2 times with 50 ml ice cold diethyl ether. The yellow powder was collected and dried overnight under vacuum, final yield was 50%.

¹H NMR (500 MHZ, DMSO-d₆): δ 7.46 (s, 1H) δ 6.73 (s, 1H) δ 3.12-2.99 (m, 4H). ¹³C NMR (500 MHZ, DMSO-d₆): § 156.49 (s) δ 145.24 (s) δ 136.31 (s) δ 127.32 (s) δ 111.14 (s) δ 39.15 (s) δ 31.56 (s). HRMS 296.25 calc for [M], 197.00 [M-H₂SO₄—H]⁻ found.

### Synthesis of Nitrodopamine-mPEG.

1.784 mmol (491 mg) MEEAA-NHS and 2.854 mmol (846 mg) nitrodopamine hemisulfate were dissolved in 25 ml dry DMF in a predried flask, resulting in a dark orange solution. The flask was sealed, flushed with argon and cooled in an ice bath. 785 μL N-methylmorpholine was added dropwise to the mixture using air-free technique, after approximately 10 minutes of stirring the solution becomes turbid. The mixture was allowed to stir 48 hour at room temperature, followed by evaporation overnight under vacuum at 40° C. to remove DMF, yielding a dark brown liquid. 40 ml 1M HCl was added to the crude and extracted 3 times with 40 ml CHCl₃, a dark brown solid is formed during the process at the liquid interface, care was taken to not allow this to enter the organic phase. The organic phase was extracted twice more with 50 ml brine, dried with Na₂SO₄ and evaporated using rotary evaporation. The resulting solid was purified with prep HPLC using a gradient from solvent A (MQ water containing 0.1% TFA) to solvent B (ACN containing 0.1% TFA), after freeze-drying the final product was isolated as a fluffy white-yellow solid with 75% yield.

¹H NMR (500 MHZ, MeOD): δ 7.54 (s, 1H) δ 6.7 (s, 1H) δ 3.93 (s, 2H) δ 3.62 (s, 4H) δ 3.61-3.58 (m, 2H) δ 3.57-3.5 (m, 4H) δ 3.35 (s, 3H) δ 3.07 (t, 2H, J=6.68 Hz). ¹³C NMR (500 MHZ, MeOD): δ 173.05 (s) δ 152.4 (s) δ 145.53 (s) δ 141.06 (s) δ 129.39 (s) δ 119.61 (s) δ 113.56 (s) δ 72.97 (s) δ 72.07 (s) δ 71.4 (s) δ 71.39 (s) δ 71.24 (s) δ 59.21 (s) δ 40.47 (s) δ 34.14 (s). HRMS 358.35 calc for [M], 357.35 [M-H]⁻ found.

### Synthesis of Nitrodopamine-PEG(4)-N₃

0.103 mmol (40 mg) NHS-PEG(4)-N₃ (15-Azido-4,7,10,13-tetraoxa-pentadecanoic acid succinimidyl ester) and 0.1648 mmol (48.8 mg) nitrodopamine hemisulfate were dissolved in 2 mL of dry DMF in a predried flask, resulting in a dark orange solution. The flask was sealed, flushed with argon, and cooled in an ice bath. Next, 45.3 μL N-methylmorpholine was added dropwise to the mixture using air-free technique. The mixture was allowed to stir 48 h at room temperature, resulting in a turbid solution containing a yellow-brown solid. The DMF was evaporated overnight under vacuum at 30° C. Next, the crude was dissolved in 3 ml Milli-Q water and diluted to 5 mL using a 1 mol*L-1 HCl solution, aiming to achieve a pH of approximately 1. The aqueous crude was extracted three times with 5 mL of CHCl₃; a dark brown solid is formed during the process at the liquid interface, and care was taken to not allow this to enter the organic phase. The organic phase was evaporated using rotary evaporation and dried overnight again under vacuum at 30° C., resulting in a brown-yellow sticky solid. The resulting solid was dissolved in 3 mL of a 50/50 ACN/Milli-Q water mixture, after removal of the insolubles via a 0.2 μm syringe filter the solution was purified with preparative HPLC using a gradient from solvent

A (Milli-Q water containing 0.1% TFA) to solvent B (ACN containing 0.1% TFA), and after being freeze-dried, the final product was isolated as a yellow-brown solid with 70% yield. 1H NMR (400 MHZ, D₂O): δ 7.66 (s, 1H), 6.82 (s, 1H), 3.76-3.44 (m, H), 3.07 (t, 2H, J=6.4 Hz), 2.45 (t, 2H, J=6.11 Hz). 13C NMR (100.6 MHZ, D₂O): δ 173.86 (s), 150.5 (s), 142.85 (s), 140.19 (s), 129.4 (s), 118.83 (s), 113.18 (s), 69.58 (s), 69.55 (s), 69.5 (s), 69.48 (s), 69.44 (s), 69.18 (s), 66.71 (s), 50.11 (s), 39.12 (s), 36.04 (s), 32.51 (s). HRMS 471.2 calcd for [M], 470.12 [M-H]-found.

### Synthesis of Hafnium Oxide Nanocrystals.

The NCs were synthesized from hafnium (IV) tert-butoxide (4.8 mmol, 2.26 g, 1.94 mL) and anhydrous benzyl alcohol (40 mL) according to Lauria et al. (ACS Nano 2013, 7 (8), 7041-7052). After synthesis, the nanocrystals were collected by adding diethyl ether (17 mL) to the reaction mixture and centrifugation (5000 rcf, 3 mins) in plastic centrifuge tubes. The sediment was washed three times with diethyl ether (17 mL). For functionalization with 2-(2-(2-methoxyethoxy) ethoxy) acetic acid, the sediment was first dispersed in 17 mL toluene resulting in a milky white turbid liquid. 335 μL 2-(2-(2-methoxyethoxy) ethoxy) acetic acid (0.2885 g; 1.62 mmol) was added followed by 30 mins of sonication, resulting in a transparent suspension with a few insolubles present. The insolubles were removed by centrifugation (5000 rcf, 5 mins) and the clear top layer was transferred to new plastic centrifuge tubes. NCs were sedimented by addition of 1:2 volume hexane (mixture of isomers), after centrifugation (5000 rcf, 5 mins) the organic top phase was removed and the NCs were resuspended in toluene. This purification step was repeated 3 more times before final resuspension in toluene. The purified NC suspension in toluene remains stable for at least 1 year. The dispersion in toluene can be dried and dispersed in ethanol, from ethanol the dispersion can be dried again and resuspended in MeOH or water.

### TEM Analysis.

Transmission electron microscopy (TEM) images (of a drop-cast suspension on a grid) were taken on a JEOL JEM-2200FS TEM with Cs corrector.

### Dynamic Light Scattering Analysis.

Dynamic light scattering (DLS) and Zeta potential measurements were conducted on a Malvern Zetasizer Ultra Dynamic Light Scattering system in backscattering mode (173°). DLS and Zeta potential measurements were performed respectively in a glass cuvettes and disposable folded capillary cells. All measurements were performed in triplicate at 25° C. after equilibrating inside the system for 240 seconds, sample concentration was tuned to achieve system attenuator values between 9-10. DLS data processing was performed using the Malvern “ZS Explorer” software using the “general purpose” analysis model, Zeta potential data processing was performed in the same software using the “monomodal” analysis model.

### UV-Vis Analysis.

UV-VIS spectra were recorded on a PerkinElmer Lambda 365.

### XRD Measurements.

X-ray diffraction (XRD) was performed on a Bruker D8 Advance with motorized anti-scatter screen and Autochanger and Bragg-Brentano θ-θ geometry (goniometer radius 280 mm).

The instrument uses the Cu Kα radiation (λ=1.54184° A) with no KB filter. The detector is a LynxEye XE-T Silicon strip Line detector with 192 channels. Samples were made by drop-casting a NC suspension on a silicium plate. The measurement was performed in the 15-60° 2θ range at a step size of 0.02° and a scan rate of 0.5°/min.

### X-Ray Scattering Analysis.

The Pair Distribution Function (PDF) measurement was conducted at beamline P21.1 at DESY in Hamburg, Germany in rapid acquisition mode, using a Varex 2D detector (2880×2880 pixels and 150×150 μm pixel size) with a sample to detector distance of 800 mm. The incident wavelength of the X-rays was λ=0.122 Å. Calibration of the experimental setup was performed using a nickel standard.

### Variable Temperature NMR Measurements.

Variable temperature ¹H NMR measurements were recorded on a Bruker Avance III NMR spectrometer operating at 600.13 MHz proton frequency, the instrument was equipped with an indirect 5-mm BBI probe. The probe is provided with self-shielded z-gradients. For experiments performed below 318K the temperature was calibrated using a methanol standard showing accuracy within +/−0.2 K. For variable temperature NMR measurements above 318 K a glycerol standard was used for calibration.

### NMR Measurements on PA-PEG Functionalized NCs.

Nuclear magnetic resonance (NMR) measurements for nanocrystal (NC) functionalizations with (2-(2-(2-hydroxyethoxy)ethoxy)ethyl)phosphonic acid (PA-PEG) were recorded on a Bruker Avance III NMR spectrometer (Titration with PA-PEG and transfer to water) operating at 600.13 MHz proton frequency. The instrument was equipped with a direct observe 5-mm BBFO smart probe (for ³¹P NMR) or with an indirect 5-mm BBI probe. Both probes are provided with self-shielded z-gradients. The experiments were performed at 298 K and the temperature was calibrated using a methanol standard showing accuracy within +/−0.2 K.

All other 1H NMR measurements for functionalization with PA-PEG were recorded at a temperature of 298 K on a Bruker Avance III HD NMR spectrometer operating at 600.13 MHZ proton frequency, the instrument was equipped with a cryogenic QCI-F probe. All other ³¹P NMR measurements for functionalization with PA-PEG were recorded at a temperature of 298 K on a Bruker Avance Neo spectrometer operating at 500.13 MHZ proton frequency, the instrument was equipped with a BBFO probehead. Probes for both spectrometers are provided with self-shielded z-gradients. The temperature was calibrated using a methanol standard showing accuracy within +/−0.2 K.

### NMR Measurements on PA-Hex-PEG and Nitrodopamine-mPEG Functionalized NCs.

Nuclear magnetic resonance (NMR) measurements for nanocrystal (NC) functionalizations with (6-(2-(2-(2-hydroxyethoxy)ethoxy)ethoxy)hexyl)phosphonic acid (PA-hex-PEG) and N-(4,5-dihydroxy-2-nitrophenethyl)-2-(2-(2-methoxyethoxy) ethoxy) acetamide (Nitrodopamine-mPEG) were recorded on a Bruker Avance III HD NMR spectrometer operating at 600.13 MHz proton frequency, the instrument was equipped with a cryogenic QCI-F probe. ³¹P NMR measurements for functionalization with PA-hex-PEG were recorded at a temperature of 298 K on a Bruker Avance Neo spectrometer operating at 500.13 MHZ proton frequency, the instrument was equipped with a BBFO probehead. Probes for both spectrometers are provided with self-shielded z-gradients. The temperature was calibrated using a methanol standard showing accuracy within +/−0.2 K.

### NMR Measurements on Synthesized Ligands.

¹H, ¹³C{¹H}, ³¹P{¹H} and 2D NMR measurements for synthesized ligands were recorded at a temperature of 298 K on a Bruker Avance Neo spectrometer operating at 500.13 MHZ proton frequency, the instrument was equipped with a BBFO probehead. The probe is provided with self-shielded z-gradients. The temperature was calibrated using a methanol standard showing accuracy within +/−0.2 K.

### NMR Experimental Parameters.

For quantitative 1D ¹H measurements, 64k data points were sampled with the spectral width set to 20 ppm and a relaxation delay of 30 s. Concentrations were obtained using the digital ERETIC method.² DOSY measurements were performed with a double stimulated echo and bipolar gradient pulses (dstebpgp2s). The gradient strength was varied quadratically from 2-95% of the probe's maximum value in 8 steps if a diffusion filtered slice was required or 32 steps if the creation of a pseudo 2D spectrum was required. The gradient pulse duration and diffusion delay were optimized to ensure a final attenuation of the signal in the final increment of less than 10% relative to the first increment. The diffusion coefficients were obtained by fitting a modified Stejskal-Tanner equation to the signal intensity decay:

$$I = {I}_{0} {e}^{- {( γ δ g )}^{2} D ( Δ - 0.6 δ )}$$

I are the signal intensities, D are the linear diffusion coefficients, γ is the gyromagnetic ratio of the studied nucleus, g is the gradient strength, δ is the pulsed field gradient duration and Δ is the diffusion delay. A correction factor of 0.6 is applied for δ due to the smoothed squared pulse shape used for the gradient pulses. For 1D ³¹P{¹H} measurements, in the zgpg30 pulse sequence 25000 data points were sampled with the spectral width set to 270.81 ppm and 4k scans, LB was set to 40 Hz during spectrum post-processing. For 1D ¹³C{¹H} measurements, in the zgpg30 pulse sequence 120480 data points were sampled with the spectral width set to 239.49 ppm and 4k scans.

### Spin Filtration Purification in MeOH and H₂O.

An NC suspension containing maximum 50 mg material dissolved in 2 ml solvent is transferred to a pre-rinsed Sartorius Vivaspin (30000 MWCO) spin-filtration tube via a 0.2 μm syringe filter. The suspension is diluted to a volume of 20 ml with MeOH or Milli-Q water, the solution was then allowed to spin in a centrifuge for 30 mins at 2100 rcf. For phosphonic acid NC functionalizations 3 spin filtration cycles were performed per sample using MeOH, for nitrodopamine-mPEG NC functionalizations a minimum of 2 spin filtration cycles using Milli-Q water were performed until the filtrate was colorless. The concentrate was collected, evaporated and suspended in (deuterated) MeOH or (deuterated) H₂O, 30 minutes of sonication was performed to ensure that all agglomerates were resuspended and to minimize insolubles.

Titration with PA-PEG and PA-Hex-PEG.

A small amount of the purified toluene NC stock suspension was evaporated to yield approximately 45 mg functionalized material. The NCs were suspended in 0.5 ml absolute EtOH and sonicated for 30 mins, after which the solvent was evaporated again. The NCs were then suspended in 0.5 ml MeOD and sonicated for 30 mins, a quantitative 1H NMR measurement was performed using the digital ERETIC method to determine the MEEAA concentration. Care was taken to subtract the MeOH solvent peak, which partly overlaps with MEEAA signals, from the calculation to ensure accurate concentration determination. Next, a stock solution containing at least 3 equivalents of either PA-PEG or PA-hex-PEG was created in MeOD. The titration was performed by addition of PA-PEG or PA-hex-PEG in steps of 0.1 eq, at each addition step the NMR tube was flipped, then mixed using vortex rotation for 2 minutes followed by sonication for a few seconds.

NC Functionalization with PA-PEG and PA-Hex-PEG.

In a typical functionalization the same method as during the titration with PA-PEG or PA-hex-PEG is used. Except here 1.5 equivalents of phosphonic acid are added all at once to the MEEAA functionalized NCs, stirred and sonicated 10 mins, followed by purification using spin filtration to yield pure PA-PEG or PA-hex-PEG functionalized NCs.

Titration with D₂O.

NCs functionalized with PA-PEG or PA-hex-PEG were purified using spin filtration as described above, the concentrate was evaporated and resuspended in 500 μL MeOD. D₂O was added in a stepwise manner to achieve a final D₂O concentration of 25, 50, 75 and 100% respectively. When required, the suspension was evaporated between measurements to achieve the desired D₂O concentration without increasing sample volume above 0.8 ml.

Titration with Nitrodopamine-mPEG

A small amount of the purified toluene NC stock suspension was evaporated to yield approximately 10 mg functionalized material. The NCs were suspended in 0.5 ml absolute EtOH and sonicated for 30 mins, after which the solvent was evaporated again. The NCs were then suspended in 0.5 ml MeOH and sonicated for 30 mins, after which the solvent was evaporated again. The NCs were suspended in 0.5 ml D₂O and a quantitative ¹H NMR measurement was performed using the digital ERETIC method to determine the MEEAA concentration. 1.5 equivalents of nitrodopamine-mPEG (compared to the amount of MEEAA on the NCs) was preactivated in D₂O by addition of 2 equivalents of NaOD (compared to the amount of nitrodopamine-mPEG required). Preactivated nitrodopamine-mPEG was added in steps of 0.5 equivalents while ensuring that pH remains above 5 during the addition, after each addition step the NMR tube was flipped, then mixed using vortex rotation for 2 minutes followed by sonication for a few seconds.

NC Functionalization with Nitrodopamine-mPEG

In a typical functionalization the same method as during the titration with nitrodopamine-mPEG is used. Except here 1.5 equivalents of preactivated nitrodopamine-mPEG are added all at once while ensuring the pH remains above 5 during the entire addition. The mixture was stirred and sonicated 10 mins, followed by purification using spin filtration to yield pure nitrodopamine-mPEG functionalized NCs. The authors note that this method is scalable to higher amounts of NCs as long as the maximum loading allowed per spin filter is not exceeded.

### Influence of pH on Ligand Binding

For all measurements 2 ml solvent was found to be the minimum required for the micro pH electrode to be able to measure pH values. A 5M NaCl stock solution was used to achieve a sample salt concentration of 0.01M, pH values were adjusted using 0.01M stock solutions of DCI and NaOD in D₂O.

Phosphonic acids: pure PA-PEG and PA-hex-PEG functionalized NCs in MeOH were created using above methods, the NC suspension was evaporated and resuspended in D₂O. ³¹P NMR measurements were performed at several pH values. The amount of bound and unbound ligands in the ³¹P spectra were quantified via a multi-peak fitting procedure (peak deconvolution).

Nitrodopamine-mPEG: pure nitrodopamine-mPEG functionalized NCs in Milli-Q water were created using above methods, the NC suspension was evaporated and resuspended in D₂O. Quantitative ¹H NMR measurements were performed at several pH values. The amount of bound and unbound ligands in the ¹H spectra were quantified via a multi-peak fitting procedure (peak deconvolution).

### Dynamic Light Scattering Stability Evaluation

For all measurements pure functionalized NCs in MeOH (for phosphonic acids) or Milli-Q water (for nitrodopamine-mPEG) were created using above methods, the NC suspensions were evaporated and resuspended in Milli-Q water. For Z-average and zeta potential measurements NC concentration was tuned to achieve system attenuator values between 9-10. 2 ml solvent was found to be the minimum required for the micro pH electrode to be able to measure pH values and to perform Z-average and zeta potential measurements. All measurements were performed in triplicate at 25° C. after equilibrating inside the system for 240 seconds.

Influence of pH on Z-average values and zeta potential. A filtered 5M NaCl stock solution was used to achieve a sample salt concentration of 0.01M, the suspensions were sonicated for 15 minutes and filtered through a 0.2 μM Supor syringe filter to remove dust before starting the titration. pH values were adjusted using 0.01M filtered stock solutions of HCl and NaOH in Milli-Q water.

Stability in 2×PBS: The suspension was filtered through a 0.2 μM Supor syringe filter, followed by pH adjustment to 7.4 using 0.01M filtered stock solutions of HCl and NaOH in Milli-Q water. PBS concentration was increased by stepwise addition of a filtered 10×PBS stock solution, pH was checked after each addition step and readjusted to 7.4 if required. Stability over time in 2×PBS measurements were performed in closed quartz cuvettes, which remained at room temperature and in closed conditions during the entire duration of the stability tests.

### CT Scanning

### General Considerations

In vitro and in vivo CT scans were acquired on a Molecubes X-cube benchtop CT scanner using the built-in high-resolution scan protocol at a tube potential of 50 kV. The acquired scans were reconstructed with a voxel size of 200, 100 or 50 μm using the scanners' built-in iterative reconstruction algorithm, no additional denoising step was applied to the data. The reconstructed data was visualized using the Amide or Horos software package, windowing level was generally set from −1000 to 1000 HU for each scan unless otherwise specified.

### In Vivo CT Scans

For the in vivo scans, the mice were anaesthesized beforehand using 5% isoflurane for induction and 2% isoflurane for maintenance inside the scanner. After induction, the mice were placed on the heated scanbed in a prone position. On average, for a full-body scan, the mice received an X-ray dose of approximately 340 mGy per scan. For the in vivo experiments reconstruction was always performed at 200 micron resolution at every scanpoint.

### CT Scans of HfO₂ NC Concentration Series in PBS

After scanning and reconstruction, the X-ray attenuation, expressed in Hounsfield Unit values, was quantified in each sample tube using a spherical region-of-interest measuring 1×1×1 mm. The median pixel value was taken during quantification to avoid possible effects from outliers. When plotting the X-ray attenuation in function of NC concentration the ligand weight (18.9 m %) was subtracted beforehand from the NC weights, as the ligands are purely organic and will provide no significant contribution to X-ray attenuation.

### Near-Infrared Fluorescence Imaging

An IVIS Lumina LT Series Ill in vivo imaging system was used to visualize NC sample and in vivo lymph node fluorescence. Band pass excitation filters at 710 (+15 nm) and 745 (+nm) and a band pass emission filter in the ICG window (810-875) nm were used to perform imaging. Fluorescence images were typically overlayed with an visible light photograph.

### Preparation of Catechol Functionalized NCs

### Nitrodopamine-mPEG Functionalized NCs

A small amount of a purified MEEAA-functionalized NC stock suspension in toluene was evaporated to yield approximately 20 mg functionalized NCs. The NCs were resuspended in deuterated benzene and the ligand concentration was determined via quantitative 1H NMR using the digital ERETIC method, providing a MEEAA concentration in terms of mol MEEAA per mg of functionalized NCs. Next, a small amount of the toluene stock suspension was evaporated to yield approximately 50 mg functionalized NCs. The NCs were resuspended in 2 ml absolute ethanol and sonicated for 30 mins, after which the solvent was evaporated again. The NCs were then suspended in 2 ml MeOH and sonicated for 30 mins, after which the solvent was evaporated again. Finally, the NCs were suspended in 2 ml endotoxin free ultrapure water and sonicated for 30 mins. For nitrodopamine-mPEG functionalized NCs, 1.2 equiv of nitrodopamine-mPEG, based on the MEEAA concentration determined via quanitative NMR, was weighed in a separate vial and dissolved in 3 ml endotoxin-free ultrapure water. 2 equiv of NaOH, compared to the amount of nitrodopamine-mPEG required, was added to the nitrodopamine-mPEG solution, resulting in a deep burgundy color. The ligand solution was then added quickly to the NC suspension under heavy stirring, a brief turbidness during the transition from acidic to basic pH can be observed and is normal, after full addition of the ligand a transparent red-orange liquid is obtained which was subsequently sonicated for 15 mins and stirred heavily. After sonication and stirring, the pH of the suspension was adjusted to approximately 9 before starting purification. A pure nitrodopamine-functionalized NC suspension was obtained after purification via spin-filtration.

Nitrodopamine-mPEG and Nitrodopamine-PEG(4)-N₃ functionalized NCs

For NCs functionalized with the mixed catechol ligand shell containing roughly 98% nitro-dopamine-mPEG and 2% nitrodopamine-PEG(4)-N₃ (about one azide per NC), the same procedure as above was followed but with a mixture of 1.18 equiv nitrodopamine-mPEG and 0.02 equiv nitrodopamine-PEG(4)-N₃ instead.

### Number of Nanocrystals and Ligand Density

To calculate an approximation of the number of NCs for a given amount of NC weight, the inventors start by calculating the molar volume (21.745 cm³/mol) for the material by dividing material density (9.68 g/cm³) by molecular weight (210.49 g/mol).

$$MV = \frac{ρ}{{MW}_{material}}$$

Next the average NC volume (15.33 nm³) is calculated based on a prolate spheroid shape, an average major axis radius of 2.52 nm and an average minor axis radius of 1.205 nm.

$${V}_{NC} = \frac{4}{3} π {r}_{major} {r}_{minor}^{2}$$

The average NC volume, converted to cm³, is then divided by the molar volume and Avogadro's constant to determine mol HfO₂/NC (7.05*10⁻²² mol/NC)

$$\frac{mol}{NC} = \frac{{V}_{NC}}{MV \cdot {N}_{A}}$$

Finally, the number of NCs (N_{NC}) is determined by dividing NC weight in grams (ligand weight subtracted) with molecular weight (210.49 g/mol) and mol HfO₂/NC (7.05*10⁻²² mol/NC)

$${N}_{NC} = \frac{{m}_{NC naked}}{MW \cdot ( \frac{mol}{NC} )}$$

To calculate ligand density on the NC surface the inventors start by calculating the average surface area of the NCs (A_{NC}, 32.42 nm²), applying the area formula for a prolate spheroid.

$${A}_{NC} = 2 π {r}_{minor}^{2} + 2 π \frac{{r}_{minor} \cdot {r}_{major}}{e} \cdot arc sin ( e ) \\ e = \frac{S}{1 - \frac{{r}_{minor}^{2}}{{r}_{major}^{2}}}$$

Next, based on TGA mass loss (m %) of for a given amount of purified functionalized NCs, the ligand amount, in mol, (n_{Ligand}, mol) is obtained.

$${n}_{Ligand} = \frac{{m}_{NC} \cdot \frac{TGA m \%}{100}}{{MW}_{Ligand}}$$

Multiplying the ligand amount (n_{Ligand}) with Avogadro's constant and dividing by the number of NCs (N_{NC}) gives ligands/NC

$$\frac{ligands}{NC} = \frac{{n}_{Ligand} \cdot {N}_{A}}{{N}_{NC}}$$

Finally, dividing ligands/NC by the NC surface (A_{NC}) gives the ligand density in nm².

$$Ligand density = \frac{\frac{ligands}{NC}}{{A}_{NC}}$$

### Spin-Filtration Purification

A 20 ml Sartorius Vivaspin (30000 MWCO) spin-filtration tube was sanitized with 70% ethanol and then prerinsed with 20 ml endotoxin-free ultrapure water at neutral pH. An NC suspension at pH 8-9 containing maximum 50 mg dissolved material was transferred to the spin-filtration tube via a 0.2 m syringe filter. The suspension is diluted to a volume of 20 ml with endotoxin-free ultrapure water at neutral pH, the solution was then allowed to spin in a centrifuge for 20 mins at 2100 rcf. For nitrodopamine-mPEG and mixed catechol ligand shell functionalizations, a minimum of 5 spin filtration cycles using endotoxin-free ultrapure water were performed until the filtrate was colorless. The concentrate was collected from the spin-filter and evaporated under vacuum at 35° C. The dried NCs can either be stored as a powder at room temperature or resuspended in water or PBS at pH 7.4 at a maximum NC concentration of approximately 300 mg HfO₂/ml. In the latter case, organic ligand weight (18.9 m %) has been subtracted from the functionalized NC weight in the concentration calculations, the suspensions are filtered via a sterile 0.2 m syringe filter and stored in sterile vials. NC suspensions in water or PBS remain stable for at least two months, even at high concentrations.

### Dye Conjugation to Functionalized NCs

In the functionalization of the NCs with the mixed catechol ligand the assumption is made that the final purified product has about 1 azide present per NC. In a typical dye coupling reaction, 10 mg of the mixed catechol functionalized NCs are first weighed in a HPLC vial and dissolved in 100 μL endotoxin-free ultrapure water at pH 7. Based on a ligand mass contribution of 18.9m % (confirmed via TGA mass loss) this corresponds to 8.11 mg naked nanocrystals, in turn corresponding to 5.4662*10¹⁶ NCs based on an average major diameter of 5.04 nm, an average minor diameter of 2.41 nm and a prolate spheroid NC shape. 1.1 equiv of IRDye-800CW-DBCO (5.4662*10¹⁶ molecules, 0.091 μmol, 0.12 mg) was weighed in a HPLC vial on a analytical balance and dissolved in 200 μL endotoxin-free ultrapure water at pH 7. Dye molecule concentration was confirmed using UV-VIS spectroscopy, applying the Lambert-Beer law with a path length of 1 cm and dye extinction coefficient of 240000 L*(mol*cm)⁻¹ with the aborbance measured at a wavelength of 774 nm, see Eq. 10.

A=c·ϵ·l

The dye solution was added to the NCs and diluted further to a volume of 300 μL, resulting in a transparent yellow-green suspension. The reaction mixture was shielded from light and stirred for 2 hours at 30° C. The NCs were then purified via 3 consecutive spin filter cycles, after which the concentrate was isolated and evaporated under vacuum at 30° C. while shielded from light. The resulting deep green NC powder, containing approximately 1 dye molecule/NC, was stored in a freezer at −20° C. under argon.

### Formulation Preparation

To achieve a formulation where NC and dye concentration are respectively 292 mg NCs/ml and 28 μmol/L, the first step in is determination of dye grafting density on the functionalized NCs, in this regard 1 mg of dried dye-functionalized NCs are suspended in 4 ml 1λPBS and measured using UV-VIS. Using the Lambert-Beer law with a path length of 1 cm, dye extinction coefficient of 240000 L*(mol*cm)⁻¹ and the absorbance measured at the maximum NIR absorption peak (+780 nm) the dye concentration is determined in mol*L⁻¹, this is then converted to mol dye/mg functionalized NC. Next, a stock solution of nitrodopamine-mPEG functionalized NCs is mixed with the dye-functionalized NC powder and sonicated for 15 mins to achieve an NC concentration of 292 mg NCs/ml and 28 μmol/L conjugated dye. To exemplify this procedure: suppose a dye-conjugated NC batch is available containing 5.796*10⁻⁹ mol dye/mg functionalized NCs. Mixing 0.199 mg (18.9 m % organic weight, 0.161 mg naked NCs) of this dye-functionalized powder with 40 μL of a nitrodopamine-mPEG NC stock at a concentration of 288 mg NCs/ml results in the desired NC and dye concentration.

### Subcutaneous Footpad Injection

Prior to subcutaneous injection with a PBS solution or NC suspension, the mice were anaesthetized using 5% isoflurane for induction, reduced to 2% isoflurane for maintenance during the injection procedure. Once anesthetized, the mice were placed in a supine position on a heated bed and the hind foot was heated for approximately 30 seconds using a infrared lamp. The footpad skin was tightened, and a 0.5 ml insuline syringe with 29 G needle was inserted subcutaneously in the heel region, advancing it approximately 3 mm towards the toes. During the entire process the needle was visible through the thin skin layer of the footpad. Next, a maximum volume of 50 μL PBS solution or NC suspension was injected slowly, simultaneously slowly retracting the needle towards the heel. After the injection was completed, the needle was kept in place in the footpad for 30 more seconds before removing it from the hind foot. Immediately after needle retraction, a generous layer of 5% xylocaine was applied to the injected hind foot as an analgesic.

1. A nanoparticle comprising
a. a nanocrystal comprising or essentially consisting of hafnium (IV) oxide (HfO₂) having a diameter equal or less than (≤) 15 nm, and
b. a plurality of dispersant molecules attached to the surface of said nanocrystal, wherein each of the dispersant molecules comprises or essentially consists of
i. a surface adsorption moiety selected from the group comprising a catechol or a gallol, particularly a 1,2-hydroxy-4-nitrophenyl moiety; and
ii. an oligo (ethyleneglycol) moiety.

2. The nanoparticle according to claim 1, wherein the nanocrystal has a diameter of ≤6 nm, particularly wherein the diameter is ≤4 nm, more particularly wherein the diameter is ≤3.5 nm.

3. The nanoparticle according to claim 1, wherein the nanocrystal is characterized by an aspect ratio of 0.5 to 0.9.

4. The nanoparticle according to claim 1, wherein the dispersant molecule has a molecular mass of ≤500 g/mol, particularly of ≤400 g/mol.

5. The nanoparticle according to claim 1, wherein the dispersant molecule comprises, particularly consists of,
i. a surface adsorption moiety selected from nitrodopamine, nitroDOPA, DOPA, dopamine, mimosine and
ii. an (CH₂—CH₂—O)ₙCH₃ moiety, with n being an integer selected from 2, 3, 4 and 5.

6. The nanoparticle according to claim 1, wherein the dispersant molecule comprises, particularly consists of

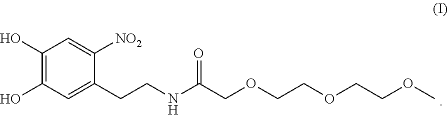 [SMILES: COCCOCCOCC(=O)NCCc1cc(O)c(O)cc1[N+](=O)[O-]]

7. The nanoparticle according to claim 1, wherein the nanoparticle comprises an additional dispersant molecule, wherein the additional dispersant molecule is selected from the group consisting of 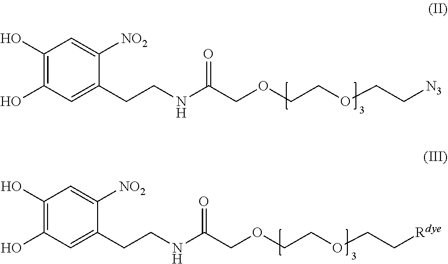
wherein R^{dye} is a fluorescent dye.

8. The nanoparticle according to claim 1, wherein the density of dispersant molecules on the nanocrystal is 0.5-5 per nm².

9. The nanoparticle according to claim 1, wherein the nanoparticle comprises a dye molecule for optical localisation.

10. A composition comprising a plurality of nanoparticles according to claim 1.

11. The composition according to claim 10, wherein the composition is a stable aqueous suspension.

12. The composition according to claim 11, wherein the composition has a pH from pH 6 to pH 10, particularly a pH ranging from pH 6.5 to pH 8.0.

13. The composition according to claim 10, wherein 80% of the nanoparticles have a diameter between 2.0 nm and 5.0 nm, particularly wherein 85% of the nanoparticles have a diameter between 2.5 nm and 4.5 nm.

14. A composition according to claim 10 for use in medicine.

15. A composition according to claim 10 for use as a radiotherapy enhancing agent (radiosensitizer).

16. A composition according to claim 10 for use as a computed tomography (CT) contrast agent.

17. A method to manufacture a composition according to claim 10, comprising
a. providing a suspension of hafnium (IV) oxide (HfO₂) nanocrystals stabilized by a carboxylic acid ligand, particularly by MEEAA, in aqueous medium;
b. adding to the suspension an alkaline solution of a dispersant molecule, wherein the dispersant molecule is composed of
i. a surface adsorption moiety comprising two aromatic hydroxide functions, the surface adsorption moiety being selected from the group comprising a catechol or a gallol, particularly a 1,2-hydroxy-4-nitrophenyl moiety; and
ii. an oligo (ethyleneglycol) moiety;
wherein both aromatic hydroxide functions are deprotonated in the alkaline solution;
c. adjusting the pH of the suspension to physiological pH;
d. optionally, isolating the composition, particularly by size exclusion/spin filtration.
